(19) United States
(12) Patent Application Publication
(10) Pub. No.: US 2004/0185058 A1
(43) Pub. Date: Sep. 23, 2004

(54) Composite superimmunogen for bi-functional vaccine use for the treatment of illnesses associated with a stromal tissue disorder

(76) Inventors: Daniel Zagury, Paris (FR); Bernard Bizzini, Albi (FR); Paul Cohen, Paris (FR); Jean Francois Zagury, Paris (FR); Helene Le Buanec, Paris (FR)

Correspondence Address: JACOBSON HOLMAN PLLC, 400 SEVENTH STREET N.W., SUITE 600, WASHINGTON, DC, 20004, US

(21) Appl. No.: 10/486,329

(22) PCT Filed: Aug. 9, 2002
(86) PCT No.: PCT/FR02/02853

(30) Foreign Application Priority Data
Aug. 10, 2001 (FR) 01/10751

(51) Int. Cl. A61K 39/00; A61K 39/12

(52) U.S. Cl. 424/185.1; 424/186.1

(57) ABSTRACT

The invention is relative to novel means of systemic or mucosal vaccinial therapy against some cancers, viral infections and allergy which are provided by the invention under the form of a family of composite superimmunogenic compounds for bifunctional vaccinial use able to induce an immune response raised towards two distinct targets, respectively, the causal pathogenic antigenic structure, on the one hand, and locally produced factors responsible for a subsequent immunotoxic or neoangiogenic stroma disorder, on the other hand.

### FIELD OF THE INVENTION

The present invention relates to preventing and treating pathologies caused by the local tissue expression of a pathogenic antigenic structure, said expression being associated with a stroma disorder of immune or vascular order leading to an immunotoxicity or to a neoangiogenesis, such pathologies encompassing some virus infections, some cancers and allergies.

It relates to developing novel preventive or curative therapeutic means, referred to as composite superimmunogens, inducing an immune reaction against both the pathogenic antigenic structure and the protein(s) causing the associated stroma tissue disorder.

### PRIOR ART

As a result of Louis Pasteur's first experimental work, it has been attempted, during the twentieth century, to understand the immunity mechanisms with a view to preparing ever higher specificity and efficiency vaccines. The first vaccines used at a large scale comprised attenuated living germs or immunogenic preparations associated with protein or membrane impurities with a poorly characterized composition and structure acting as immunity adjuvants.

Subsequently, vaccines were developed from purified antigens, such as protein sub-unities or protein toxoids, associated with better defined, more efficient and toxicity free immunity adjuvants, essentially designed for overcoming and controlling infectious diseases through immune reaction screening towards the causal infectious agent.

During the last two decades, the large scale vaccine success, including by means of genetic engineering techniques, as well as a better understanding of the immune reaction mechanisms, has made it possible for the researchers to spread the use of vaccination with a view to treating chronic diseases associated with biological structures (germs, inert or living environment cells or particles) carrying foreign antigens or abnormally expressed antigens, such as pathologies for AIDS, cancers, allergy and auto-immune diseases.

In order to prevent or treat the above-mentioned pathologies using vaccination, it has systematically been attempted to induce an immune reaction targeted to the pathogenic structure, for example, viral proteins selectively expressed by virus-infected cells, proteins selectively expressed by cancer cells or also allergenic proteins, which are the primary causal agents for such diseases.

By way of illustration, the candidate vaccines being currently prepared in order to overcome the AIDS virus infection, more particularly the HIV1, aim at causing the induction of an immune reaction exclusively targeted towards some viral proteins or peptides.

Similarly, anti-cancer vaccines being the object of the most advanced clinical studies aim at inducing an immune reaction exclusively targeting the destruction of cells expressing antigens associated with cancer, such as viral proteins in the case of cancers caused by some papillomaviruses, or the destruction of cells infected by a virus, such as HIV1 in the AIDS disease.

According to an identical vaccin strategy, the current anti-allergy vaccines exclusively aim at inducing an immune reaction targeted onto the primary causal allergen.

Cancers are proliferations of cells which are subsequently able to spread into the body so as to form metastases. It is known that the immune system of a normal individual regularly eliminates the incipient cancer cells and the generation of a cancer is associated (1) with escaping from the local immune monitoring system followed, at an advanced stage of the cancer, by a systemic immunosuppression and (2) a proliferation of vascular endothelial cells ensuring the nutritive supply to the tumor cells, such a proliferation of the tumor cells being called neoangiogenesis.

The escaping from the host cell immune defense phenomenon by induction of their in situ paralysis is a strategy being used by numerous cancers and is necessary for their survival. Initially, the immunosuppression remains localized at the tumor level, as the individual is still able to defend himself towards the other agressions such as infections.

However, at a later stage, such an immunosuppression may spread, become generalized, as can be seen from the metastasis spread and the high vulnerability of the cancerous patient towards infections. Such an immunosuppression involves paralyzing factors being produced by the cancer cells or by cells from their environment. The local paralysis of the cells of the immune system, or immunosuppression, thus represents a major weapon for the cancer cells allowing them to escape from the host immune system. This very immunosuppression strategy is also used by the viral aggressor, in some infectious diseases, such as AIDS. Thus, proteins released by HIV1 infected cells act as true toxins on the surrounding immune cells, disturbing them and blocking in situ, i.e. in a paracrine way, the immune system cells, protecting the infected cells, the virus replication and their dissemination.

Applicant's prior work, mentioned in the published international application n^{o}WO 00/03732, showed that in the case of ATL leukemia, neck of the womb cancer, and Kaposi's sarcoma, respectively, three proteins were involved in a local immunosuppression at the level of the tumors or the VIH1 infected cells:

the Tax protein of the HTLV 1 virus,

the E7 protein of the papillomavirus, and

the Tat protein of the VIH-1 virus.

The Applicant also disclosed that some of those immunosuppressive proteins, such as the Tat protein of HIV1 and the protein E7 of HPV (strains 16 and 18) also have activating effects on the vascular endothelial cells.

They consequently suggested the development of anti-cancer or anti-virus vaccines comprising a detoxicated derived immunogenic compound from a protein originating from cancer cells, from cells infected by a virus or from stroma immune cells, initially immunosuppressive and/or angiogenic with a local action, such as for example, a protein derived from the Tat protein of the HIV1 virus, the Tax protein of a HTLV1 virus, the E7 protein of a papillomavirus as well as a mannan-dependent lectin, under an inactivated form.

In another aspect of a potential therapeutic strategy for overcoming AIDS, cancer and allergies, based on a principle similar to that of the vaccines provided in the PCT application n^{o} WO 00/03732 above, ZAGURY D et al. (2001, Proc. Natl. Acad. Sci. USA, 98(14): 8024-8029), in a bibliographical study, suggest to induce some anti-cytokin immunity in patients in order to counterbalance the abnormal production in such pathologies of some cytokins, including interleukins, lymphokins, monokins, interferons, which physiologically act in the tissues, locally as a factor of cell proliferation, differentiation or programmed death.

The above-mentioned authors state that the vaccinial therapeutic strategies were, until today, exclusively targeted on the antigenic aggressor, whether it is a microorganism, a cell or an allergen, but never tried to overcome the disturbance of cytokins induced under the effect of the aggressor. These authors offer a traditional vaccination the aim of which would be to neutralize or block the immunotoxic effects of the stroma, and to allow for the normal progress of the immune reaction adapted towards the antigenic aggressor. However, ZAGURY et al. (2001) do not offer any concrete experimental evidence able to show the benefit that inducing such an immune response would offer to virus infected patients, cancerous patients as well as individuals prone to severe, for example systemic, allergic reactions.

There is a need in the state of the art for means allowing for an improved vaccinial therapy, both safe and more efficient that the current vaccinial therapy, in order to prevent or treat some cancers, viral infections, including by the HTLV-1 or HIV-1 viruses, or severe allergies.

### SUMMARY OF THE INVENTION

Such novel means of systemic or mucosal vaccinial therapy against some cancers, viral infections and allergy are provided by the invention under the form of a family of composite superimmunogen compounds for bifunctional vaccinial use capable of an immune response directed towards two distinct targets, respectively, the causal pathogenic antigenic structure, on the one hand, and locally produced factors responsible for a subsequent immunotoxic or neoangiogenic stroma disorder.

An object of the invention is a bifunctional composite superimmunogen comprising two distinct immunogenic polypeptides, physically linked one to the other, both polypeptides respectively consisting in:

(a) a first immunogenic polypeptide inducing a cell immune reaction, or a cell and humoral immune reaction, towards an inert or a living cell, microbial, or particle pathogenic antigenic structure;

(b) a second immunogenic polypeptide inducing the production of neutralizing or blocking antibodies towards a local circulating protein of the stroma selected amongst a cytokine factor or a cell regulation factor with immunotoxic or angiogenic properties, such a factor able either to be produced by cancer cells, virus infected cells or stroma cells, including the lymphocytes T and cells having the antigen (APC), or to be induced by pathogenic, including allergenic, particle structures,

for obtaining a drug with an anti-cancer, anti-viral or anti-allergic action inducing a mucosal or systemic immunity both towards the pathogenic antigenic structure and the local circulating protein of the stroma.

In a first aspect, the polypeptide (a) induces a cell immune reaction, and optionally, also a humoral immune reaction, towards an antigen specifically expressed by cancer cells, virus infected cells or an antigen component of particule structures, including living particle structures, such as pollen, acarids and some parasites such as Leishmania major, and inert particle structures, such as dust or cat's hairs.

The first immunogenic polypeptide (a) is selected amongst (i) an immunogenic protein selectively expressed by cancer cells, selectively expressed by virus infected cells or constitutive of an allergenic pathogenic structure, if need be detoxicated, and (ii) a protein derived from the protein (i).

In a second aspect, the polypeptide (b) induces a humoral reaction towards the circulating protein locally released in the tissue stroma in an abnormal way

In this second aspect, the immunogenic polypeptide (b) is selected amongst (i) the local circulating protein of the stroma, if need be, detoxicated, and (ii) a protein derived from protein (i).

It is stated that some proteins with immunotoxic or angiogenic properties, which are components of cell pathogenic structures and which are released in the stroma, could be used as a polypeptide (a) or a polypeptide (b) of a composite superimmunogen. This is the case for the Tat protein of HIV, the E7 protein of HPV as well as the cell regulation factor p53.

Preferably, the following superimmunogen compounds are used:

a) Anti-Viral Vaccination

the composite superimmunogen (a) gp160-(b) Tat toxoid of HIV1;

the composite superimmunogen (b) Tat peptide [1-15; 46-60]-(a) gp160 of HIV1;

the composite superimmunogen (a) Tat toxoid of HIV1—(b) IFNα;

the composite superimmunogen (a) Tat toxoid—(b) Tat peptide [1-15;46-60] of HIV1;

b) Anti-Cancer Vaccination

the composite superimmunogen (a) L1—(b) E7 of HPV;

the composite superimmunogen (a) E7 of HPV—(b) VEGF;

c) Anti-Allergy Vaccination

the composite superimmunogen (a) Betv1a—(b) IL4 heterolog

According to the invention, the polypeptides (a) and (b) are physically linked one to another in that they are, in all cases, presented together with the cells of the immune system on a single carrier. The polypeptides (a) and (b) may be covalently linked on the same molecular structure.

The polypeptids (a) and (b) may also be both included within a single physical structure for example on monoparticles with a diameter between 10 and 500 nanometers, preferably 10 and 1000 nanometers and most preferably between 10 and 100 nanometers, for example, IMS nanoparticles, as disclosed, for example, by Aucouturier et al. (2001), of chitosan, as disclosed, for example, by Sjaugrud et al. (1999), of liposomes, or biodegradable particles such polylactid acid (PLA), poly-ε-caprolactone (PCL) or poly(lactide-coglycolid) (PLG) disclosed by Baras et al. (1999).

In a first aspect, the use is characterized in that the polypeptides (a) and (b) are directly linked to one another covalently.

In a second aspect, the use is characterized in that the polypeptides (a) and (b) are separated from one another, with the peptidic superimmogen, by a spacer chain. The spacer chain may for example consist in a linear spacer peptide, a branched spacer peptide as well as a bifunctional spacer compound such as SMCC or SIAB.

In a third aspect, the polypeptides (a) and (b) are immobilized on nanoparticles or embedded within microparticles or within nanoparticles. Preferably, the polypeptide (a) and the polypeptide (b) are immobilized both on the same nanoparticle, or embedded within the same nanoparticle.

The invention is also relative to an immunogenic peptidic conjugate comprising two distinct polypeptides, linked to one another, both polypeptides respectively consist in polypeptides (a) and (b) as previously defined.

It is also relative to a nucleic acid coding a composite superimmogen compound such as hereinabove defined, as well as an expression cassette and a recombinant vector comprising such a nucleic acid, as well as the use of such a nucleic acid, such an expression cassette or such a recombinant vector for obtaining a drug with an anti-cancer, anti-viral or anti-allergy action.

It also relates to an immunogenic composition comprising an immunologically efficient amount of an immunogenic compound such as defined hereinabove, in association with one or more excipients, including physiologically compatible immunity adjuvants.

Depending on the objectives being sought, systemic or mucosal adjuvants are being used. For example, a mucosal adjuvant is preferably used for preventing the cancers of the epithelial tissues and systemic adjuvants are preferably used for preventing or treating infections by viruses, such as by HIV1 and HTLV1, as well as for preventing or treating allergies.

Amongst systemic adjuvants, will be preferably used adjuvants of the IFA type (Freund's Incomplete Adjuvant), calcium phosphate or alumina hydroxide.

Amongst mucosal adjuvants, are preferably used adjuvants such as choleratoxin B (CTB) or a mutant of the labile toxin of Escherichia coli LT (LTμ).

It also relates to a mucosal or systemic vaccine, characterized in that it comprises, as an active principle, an immunogenic compound such as defined hereinabove, in association with one or more excipients, including physiologically compatible immunity adjuvants.

Another object of the invention is also an immunogenic composition and a vaccine for mucosal or systemic purposes, characterized in that they comprise a therapeutically efficient amount of a nucleic acid, of an expression cassette or a recombinant vector such as defined hereinabove.

### DESCRIPTION OF THE SINGLE FIGURE

FIG. 1 is a general scheme for the vaccinial strategy developed using composite superimmogenic compounds.

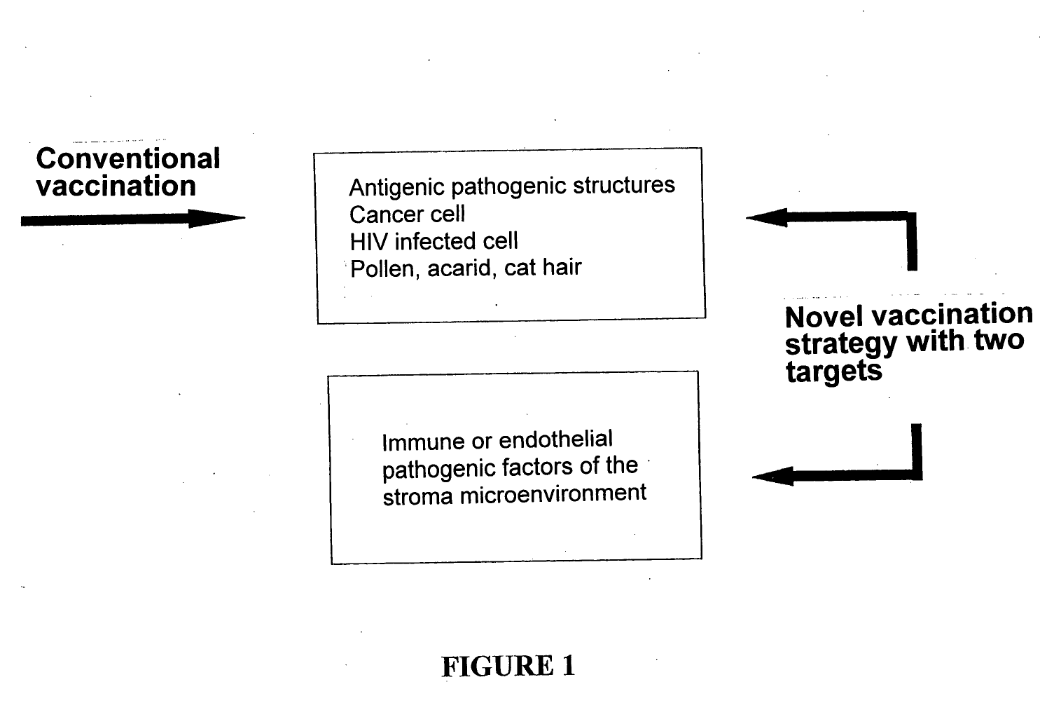

The arrow shown on the left hand side of FIG. 1 shows the nature of the structures aimed at by the traditional anti-cancer, anti-viral and anti-allergy vaccinial preparations, i.e. the pathogenic antigenic structures representing the primary causal agents for the development of the pathology, such as cancer cells, HIV infected cells or pollen allergenic antigens.

The dual arrow shown on the right hand side of FIG. 1 shows the nature of the structures aimed at by the vaccinial preparations according to the invention, which are of two types:

a) pathogenic antigenic structures such as detained hereinabove; and

b) protein factors circulating in the stroma micro-environment responsible for the immune and vascular type tissue stroma disorder observed in some cancers, severe viral or allergic infections.

### DETAILED DESCRIPTION OF THE INVENTION

It has been found out by the Applicant that the immunosuppression and the angiogenesis of the micro-environment of cells being infected by some viruses such as VIH-1 and of the micro-environment of cancer cells offer a rational explanation for the lack of efficiency of the vaccinial strategies vaccinales of the state of the art, exclusively targeting the cancer cell and not the disturbance of its micro-environment.

The Applicant's work showed that soluble factors secreted by VIH-1 infected cells, in particular Tat protein or immune cells in VIH infected patients, in particular the IFN-α and TGF-β, or produced by cancer cells, such as the E7 protein of the HPV in the neck of the womb cancer or the Tax protein of the HTLV1 in ATL leukemias, have immunosuppressive properties able to inhibit cell immune reactions within the infected tissues or the tumors and, thereby, to explain a lack of efficiency of the traditional vaccines, which exclusively use immunogens carried by pathogenic antigenic structures, such as cancer cells, VIH infected cells.

The bibliographical study has made it possible to back up the Applicant's observations, confirming the presence of immunosuppressive factors released in the extracell medium of malignant tumors.

Some of those factors, not identified as yet, were produced by:

colorectal cancer cells (Ebert E C, Roberts Al, O’Connell S M, Robertson F M, Nagase H. Characterization of an immunosuppressive factor derived from colon cancer cells. J. Immune ol. (1987) 138.2161-8 or Remacle-Bonn and M M, Pommier F J, Kaplanski S, Rance R J, Depieds R C. Inhibition of normal allogenic lymphocyte mitogenesis by a soluble inhibitor extracted from human colonic carcinoma J. Immunol. (1976) 117:1145-51,

glioblastoma cells (29-Fontana A, Hengartner H, de Tribolet N, Weber E. Glioblastoma cells release interleukin 1 and factors inhibiting interleukin 2-mediated effects. J. Immunol. (1984) 132:1837-44),

melanomas (30.Hersey P. Bindon, Czerniecki M, spurling A, Wass J, McCarthy W H. Inhibition of interleukin 2 production by factors released from tumor cells J. Immunol. (1983) 131:2837-42), or

malignant ascites (Tamura K, Shibata Y, Matsuda Y, Ishida N. Isolation and characterization of an immunosuppressive acidic protein from ascitic fluids of cancer patients. Cancer Res. (1981) 41:3244-52, Oh S K, Moolten F L. Non specific immunosuppressive factors in malignant ascites: further characterization and possible relationship to erythrocyte receptors of human peripheral Moolten F L. Non specific immunosuppressive factors in malignant ascites: further characterisation and possible relationship to erythrocyte receptors of human peripheral T cells. J. Immunol (1981) 127:2300-7).

Other transcriptional regulation factors, such as discussed hereinabove, are from cell origin, such as p53 protein, accumulated in some malignant tumors, more particularly, colorectal (Remvikos Y. Tominaga O, Hammel P, Laurent-Puig P, Salmon R J, Dutrillaux B, Thomas G. Increased p53 protein content of colorectal tumours correlates with poor survival. Br J. Cancer 1992 66:758-64, Gan H, Ouyang Q, Wang Y. Expression of p53 protein in colorectal cancer and its relationship to cell proliferative activity and prognosis. Chung Hua Chung Liu Tsa Chih (1996). The p53 protein released through active transport through secretion routes not using the peptidic signal or through passive diffusion is present in the extracellul medium, and it has been isolated through chromatography on a glass fibre, from the serum of cancerous patients (Zusman I, Sandler B, Gurevich P, zusman R, Smirnoff P, Tendler Y, Bass D, Shani A, Idelevich E, Pfefferman R, Davidovich B, Huszar M, Glick J. Comparative study of the role of serum levels of p53 antigen and its tumor cell concentration in colon cancer detection. Hum Antibodies Hybridomas. (1996): 123-8, Sandler B, Smirnoff P, Tendelr Y, Zinder O, Zusman R, Zusman I. Specificity of polyclonal anti-p53 IgG for isolation of the soluble p53 antigen from human serum. Int J. Mol. Med. 1998 1:767-70).

Cytokins, such as the notoriously immunosuppressive TGFβ, the angiogenic growth VEGF facteur, the proinflammatoriy IL-6 or IL10 also immunosuppressive, are abnormally secreted and released in the extracell medium of some cancer cells. The Applicant himself showed that cells from SIHA cancer lineage, as well as the DU145 cells of prostate cancer and the MT2 cells from the leukemic lineages abnormally produce and release in the extracell medium cytokins such as VEGF and/or IL6 whereas the RAJI cells of the leukemic lineages secrete in the extracell medium of IL10.

It is nowadays shown according to the invention that, for preventing or treating some cancers, some viral infections and allergies, more particularly severe allergies involving an anaphylactic shock risk, it is essential to induce an immune reaction simultaneously:

a) towards an antigenic, pathogenic, cellular, microbial or particulate structure being the primary causal agent of the disease; et

b) towards a local circulating protein of the stroma responible for tissue stroma disturbance, of immune or vascular order associated with the disease.

It is shown according to the invention that the above-defined immune reaction could be induced according to the invention, at a high stimulation level of the immune system cells, using a family of compounds referred to as composite superimmogens, which are bifunctionals comprising, under a physically linked to one another form, a first polypeptide inducing a immune reaction against the pathogenic antigenic structure and a second polypeptide inducing an immune reaction towards the local circulating protein of the stroma.

It is meant by <<stroma>>, for a given tissue, the intercell spaces representing the micro-environment of the dedicated cellules of a tissue. In the hepatic tissue, the stroma surrounding the cordons of the dedicated hepatic cells contains a nutritive medium, also referred to as lympha, hosting both immune cells (lymphocytes T and B, cells with the antigen or APC), polynuclears, mastocytes, fibroblasts and capillary vessels lined with endothelial cells. In the mucosal or cutaneous epithelial tissues, the sub-epithelial stroma, called dermis, also comprises intercell spaces being fixed to the connective tissue being the matrix thereof and where also circulate cells of the immune system and the above-mentioned capillary vessels.

In order to prevent or treat some cancers, the composite superimmunogens of the invention simultaneously induce:

a) an immune reaction of the cell type, through the activation of T auxiliary cells (<<T helper>> or <<Th>> cells) and/or production of T cytotoxic cells (CTL) specific for the pathogenic antigenic structure expressed by the cancer cells, for example antigens of TAA or TSA tumours, as well as the production of killing cells the so-called <<Natural Killer>> or <<NK>> allowing to selectively kill said cancer cells; and

b) a humoral immune reaction, through the induction of the production of neutralizing or blocking antibodies specific of an abnormally produced stroma circulating protein, primarily a protein with immunotoxic properties or with angiogenic properties.

In order to prevent or to treat some viral infections, if need be, associated with the development of cancers, the composite superimmogens of the invention simultaneously induce:

a) an immune reaction of cell type, through the activation of auxiliary T cells and/or through the induction of the production of cytotoxic T cells (CTL) specific of the viral pathogenic antigenic structure expressed by virus infected cells, such as some HIV1 or papillomavirus proteins, or the production of killing cells also referred to as <<Natural Killer>> or <<NK>> allowing to selectively kill said virus infected cells; and

b) a humoral immune reaction, through the induction of the production of neutralizing or blocking antibodies specific of an abnormally produced strom local circulating protein, including a viral circulating protein or a cytokin, mainly a protein with immunosuppressive or apotogenic (immunotoxic) properties or with angiogenic properties.

Moreover, the Applicant has correlated the data relative to the observations of some immunotoxicity in the case of VIH1 virus or HTLV1 virus infections with the observations of deviations of immune reactions induced by the abnormal production of the IL4 cytokin factor during immuneisations directed against particle structures of pollen, acarids or parasite structures, such as the Leishmania major which were reported by Sadicketal. (1990, J. Exp. Med., 171:115-127).

In order to prevent or treat allergies, especially severe allergies, the composite superimmogens of the invention simultaneously induce:

a) a cell immune reaction through the activation of auxiliary T cells and a humoral immune reaction, through the induction of antibodies specific of the allergenic pathogenic structure making up the allergenic particle.

b) a humoral immune reaction, through the induction of the production of neutralizing or blocking antibodies specific of an abnormally produced strom circulating protein, mainly the IL4 cytokin factor synthetized in particular by the T lymphocytes of the Th2 type. The overproduction of IL4, which is a co-stimulus of the IgE production by the B lymphocytes, is also immunotoxic in that it induces pathogenic immune responses, of the allergic inflammatory type.

An object of the invention is the use of a composite superimmunogen comprising two distinct immunogenic polypeptides, physically linked to one another, both polypeptides respectively comprising:

(a) a first immunogenic polypeptide inducing a cell immune reaction or a cell and humoral immune reaction, towards an inert or living cell, microbial or particle pathogenic antigenic structure;

(b) a second immunogenic polypeptide inducting the production of neutralizing or blocking antibodies towards a stroma local circulating protein selected amongst a cytokin factor or a cell regulation factor with immunotoxic or angiogenic properties, such a factor being either produced by cancer cells, virus infected cells or stroma cells, includiding T lymphocytes and cells having the antigen (APC), or induced by pathogenic particle structures, more specially allergenic.

The composite superimmunogen used in the invention is referred to as <<bifunctional>> as both polypeptides (a) and (b), which are the two main parts making it up, allow for the simultaneous induction of an immune reaction directed against two distinct targets, respectively the pathogenic antigenic structure and the stroma local circulating protein. However, a bifunctional composite superimmogen according to the invention could comprise in its structure a plurality of copies respectively of a polypeptide (a) and/or of a polypeptide (b).

The polypeptide (a) and the polypeptide (b) making up a composite superimmunogenic compound of the invention are referred to as <<physically linked>> to one another as they are in all cases both included in the same physical structure, molecule or particle (microparticle or nanoparticle), within which they are somewhat spaced apart from each other. As they are physically linked to one another in the composite superimmunogen, the polypeptide (a) and the polypeptide (b) are present together with the same immunocompetent cells, both the macrophages and the T or B lymphocytes.

It is meant under <<cell>> immune reaction, an activation:

either of the auxiliary T lymphocytes (Th) having a receptor specifically recognizing the polypeptide (a) or the polypeptide (b) in association with a Class II antigen of the major histocompatibility Complex (CMH);

or of the cytotoxic T lymphocytes (CTL) having a receptor at the antigen specically recognizing the polypeptide (a) in association with a Class I antigen of the MHC, and optionally of NK cells.

The cell reaction can be measured in vitro with mouse by the production of chemokins (MIP1α and MIP1β) by splenocytes and by the proliferation of such active splenocytes in the presence of such immunogens, or also by the increase in the production of IFNγ by such cells, illustrating the cytotoxic T activity.

It meant under <<humoral>> immune reaction, the production of antibodies specifically towards the polypeptide (a) or the polypeptide (b) making up the composite superimmunogen.

It is meant under <<cellular, microbial or particulate pathogenic antigenic structure>>:

either a cancer cell selectively expressing an antigen, which can be referred to as <<tumor antigen>>, and which can be killed by a cell immune reaction, and preferably by cytotoxic cells (CTL) specifically recognizing the antigen selectively carried by such a cancer cell, or which can be killed by activated NK cells;

or a virus infected cells selectively expressing a viral antigen, and which can be killed by a cell immune reaction, and preferably by cytotoxic cells (CTL) specifically recognizing the antigen selectively expressed by such an infected cell, or which can be killed by activated NK cells;

or an allergenic particle comprising an allergenic antigen which can be blocked or inactivated by an antibody specifically directed against such an allergenic antigen, said antibody being produced during a humoral immune reaction. The allergenic particles encompassing the living allergenic particle, such as pollens or parasites such as acarids or the Leishmania major, and inert allergenic particles, such as dust and cat hair particles.

The proteins <<with immunotoxic properties >> abnormally released in the stroma encompass:

proteins with immunosuppressive properties, such as the Tat protein of HIV1, the E7 protein of papillomavirus, the alpha interferon (IFNα), the TGFβ or IL10, the p53 protein under its circulating form or also the Fas ligand apoptogenic cytokine (FasI);

proteins deviating the immune reaction towards deleterious action, such as the IL4 cytokin factors stimulating the production of IgE isotope antibodies, responsible, amongst others, for the release of histamine by the mastocytes, likely to lead to a strong inflammatory deleterious release, even mortal for the organism, under the form of an anaphylactic shock.

Proteins <<with angiogenic properties>> are proteins inducing the multiplication of endothelial cells, such as the VEGF cytokin factor. In the case of cancer cell tumors, the release of proteins with angiogenic propertie, locally, in the stroma surrounding the tumor, induces some proliferation of the vascular endothelial cells during an event referred to as <<neoangiogenen>> which will allow for a vascularization of the tumor necessary for the survival thereof, more particularly through the supply of nutrients required for the viability and the multiplication of cancer cells making up the tumor.

It is meant under <<mucosal>> immunity, the induction of a humoral immune reaction leading to the production mainly of IgA isotope antibodies produced by the B lymphocytes localized at the level of the epithelial tissues, referred to as secretory IgA antibodies (s-IgA) and of a cell immune reaction within the ganglions draining the mucous membranes (mesenteric ganglions for the intestines, iliac ganglions for the vagina).

It is meant under <<systemic>> immunity, the induction of a humoral reaction leading to the production primarily of IgG isotope antibodies, systemically circulating, more particularly through the seric route and of a cell immune reaction within the peripheral ganglions or the spleen.

It is shown according to the invention that, for various composite superimmunogenic compounds such as defined hereinabove, the immune response level obtained in vivo is much higher than the immune response as observed after administration of each of the polypeptides (a) and (b), not physically linked to one another.

Without wishing to be bound by any theory, the Applicants think that the polypeptide (a) brings, within a composite superimmunogen, auxiliary T epitopes which are going to stimulate a <<helper>> effect and thereby activate, or increase the activation of the humoral immune reaction towards the polypeptide (b). Such an auxiliary action of the immunogenic polypeptide (a) is particularly required for inducing the production of antibodies directed against the polypeptide (b), when the polypeptide (b) is an autologous cytokine factor, such as for example the human IL4 within a composite immunogen designed for the administration in man.

It is shown according to the invention that the production of antibodies of the IgG type directed against the native Tat protein is twice higher when the inactivated gp160-Tat peptidic conjugate (toxoid) is administered in vivo than as a response to the administration of the inactivated Tat protein (toxoid) under a free form. Moreover, the antibodies having their production induced by the gp160-Tat composite superimmunogen (toxoid) allow to neutralise 100% of the activity of the native Tat protein, while a maximum neutralization of 75% of the Tat protein activity is observed with antibodies produced after administration of the Tat protein (toxoid) under free form.

Also, the results from the examples show that the splenocytes of animals immunized with the gp160-Tat composite superimmunogen (toxoid) produce a higher level of MIP1α and MIP1β chemiokins than the cells of animals immuneized by the Tat(toxoid), showing that an optimum stimulation of the immune cells is obtained with a composite superimmunogen according to the invention.

The splenocytes of animal immunized with the gp160-Tat composite superimmunogen (toxoid) also produce a high amount of interferon-γ when they are activated, in vitro, by the native Tat protein.

Similar results were obtained with other superimmunogens according to the invention, such as the E7-SIAB-VEGF conjugate, or also the Betv1a-IL4 conjugate.

Thus, using a composite superimmunogen such as defined hereinabove can, at least in some cases, potentialize the therapeutic effect which would be observed by separately using each of the polypeptides (a) and (b) or also by using an association of polypeptides (a) and (b) not physically linked to one another, and this at least for two reasons:

(i) the induction of an efficient immune reponse, of the cell or humoral type depending on the case, towards an antigen expressed by the tumor cells or of an antigen expressed by virus infected cell, because of the decrease or the simultaneous blockage of an immunosuppression, an apoptosis of immune cells or a proliferation of vascular endothelial cells, or also the induction of an efficient immune response not deleterious towards an allergen, because of the decrease or the blockage of the isotypic switch towards the production of igE antibodies induced by IL4;

(ii) in numerous cases, the observation of a potentialization of the immune response towards one of the immunogenic polypeptides present in the peptidic conjugate, because of the joint presentation of the second immunogenic polypeptide, carrying new auxiliary sites, to the cells of the immune system, as can be seen for example in the case of the gp160-Tat composite superimmunogen(toxoid).

### Composite Superimmunogens Used for Treating Some Cancers and Some Viral Infections

According to a first essential feature of a composite superimmunogen used according to the invention, the first immunogenic polypeptide (a) is selected amongst (i) an immunogenic protein selectively expressed by cancer cells, selectively expressed by virus infected cells or making up an allergenic pathogenic antigenic structure, if need be detoxicated, and (ii) a protein derived from protein (i).

According to a second essential feature of a composite superimmunogen used according to the invention, the immunogenic polypeptide (b) is selected amongst (i) the stroma local circulating protein, if need be detoxicated, and (ii) a protein derived from protein (i).

When the immunogenic protein selectively expressed by cancer cells, selectively expressed by virus infected cells or making up an allergenic pathogenic antigenic structure is <<toxic>>, for example <<immunotoxic>>, or when the stroma local circulating protein is <<toxic>>, for example <<immunotoxic>>, it will not be included per se as polypeptide (a) or polypeptide (b) of a composite superimmunogen according to the invention, without previously reducing or blocking its toxicity, for example its immunotoxicity while maintaining its immunogenicity.

The immunogenic protein is initially <<toxic>> when its administration to humans or animals leads to severe deleterious effects on said man's or animal's health, for example either inducing an immunotoxicity paralyzing or deviating the defense immune reactions.

The immunogenic protein is initially <<immunotoxic>> when its administration to humans or animals leads to one of the following effects:

an immunosuppression, including the apoptosis of immune cells, as is the case for the Tat proteins of HIV1, E7 of the papillomavirus, the IFNα, TGFβ or IL10 cytokin factors, or also the p53 cell regulating factor, under its circulating form in the stroma;

a deviation of the humoral immune reaction, for example inducing an isotypic switch (<<switch>>) of the B lymphocytes leading to the production of IgE., as is the case for IL4.

The initially toxic immunogenic, for example immunotoxic, protein could be detoxicated modifying it, more particulary physically, chemically or using genetic engineering techniques, as is explained in further detail hereinafter in the specicification.

The techniques, particularly chemical, allowing to detoxicate a polypeptide (a) and/or a polypeptide (b) could be implemented for preparing a polypeptide (a) or a polypeptide (b) from a protein which is not initially toxic or immunotoxic, because such treatments could also allow to stabilize the polypeptide for a better vaccine preservation.

It is essential that, similarly to toxoids, detoxicated toxins from antitetanic antitenaic or antidiphtheric vaccines, the detoxicated protein retains its immunogenicity, i.e. its ability to induce:

either a cell, or cell and humoral, immune reaction, towards the pathogenic antigenic structure, when the detoxicated protein is prepared from the immunogenic protein selectively expressed by cancer cells, selectively expressed by virus infected cells, or making up an allergenic pathogenic structure;

or a humoral immune reaction towards the stroma local circulating protein such as previously defined, when the detoxicated protein is prepared from such a stroma circulating protein.

It is meant under <<derived>> or <<to derive>>, from an immunogenic protein contained inside a targeted pathogenic antigenic structure or the targeted stroma local circulating protein, the fact that the immunogenic polypeptide (a) or (b) can be made from a peptidic fragment included in the initial immunogenic protein or also that the polypeptide (a) or (b) comprises one or more substitutions, deletions or additions of amino acids, relative to the amino acid sequence of the initial immunogenic protein, as this will be further explained in the specification. In every case, a polypeptide (a) or (b) which <<derives>> from the initial immunogenic protein maintains its ability to induce an immune reaction towards the targeted pathogenic antigenic structure ou the targeted stroma local circulating protein. When the initial immunogenic protein is <<toxic>>, the polypeptide (a) or (b) deriving therefrom has a reduced toxicity or has lost its toxicity, either because it only is a non toxic fragment of the initial immunogenic protein, or it comprises amino acid modifications, relative to the amino acid sequence of the initial immunogenic protein, reducing or blocking its toxic properties.

According to a first embodiment of a composite superimmunogen used according to the invention for preventing or treating some cancers or some viral infections, said composite superimmunogen is characterized in that the polypeptide (a) induces a cell immune reaction towards an antigen specifically expressed by cancer cells or by virus infected cells.

It is meant under <<induction of a cell immune reaction>>, the fact that the polypeptide (a), within the composite superimmunogen structure, is useful for initiating an efficient immune response, which, in the case of cancer cells or of virus infected cells, mainly consist in the production either of cytotoxic cells (CTL) specifically recognizing the targeted pathogenic antigenic structure, for example a tumoral antigen or a viral antigen, which is presented at the surface of the cancer cell or of the infected cell under a form associated with Class I antigens of the histocompatibility major complex (MHC), or of NK killer cells, preferably destroying cancer cells.

Moreover, the cell immune reaction induced by the immunogenic polypeptide (a) produces an activation of the auxiliary T cells (<<T helper>>) increasing or allowing to induce the humoral reaction based on the immunogenic polypeptide (b) and specifically directed towards the stroma circulating protein. This does not mean that a humoral immune reaction towards the polypeptide (a) is not induced. However, the induction of a humoral response towards the polypeptide (a) in the case of cancer and of virus infections, if it occurs together with the induction of the cell immune reaction, is not the objective being sought.

Preferably, the antigen specifically expressed by cancer cells of the neck of the womb cancer (HUCC) or by HIV infected cells or by ATL leukemic cells is respectively selected from (i) L1, L2 and E7 proteins of the papillomavirus, (ii) gp160, p24, p17, Nef and Tat proteins of the HIV-1 virus, (iii) Tax protein, (iv) gp61 protein or also gag proteins of the HTLV1 or 2 viruses, the protein being, if need be, detoxicated or also a protein being derived therefrom.

The polypeptide (a) is preferably selected amongst immunogenic proteins of the HIV1, immunogenic fragments of such proteins or a protein being derived therefrom.

Preferably, the first immunogenic polypeptide (a) is selected amongst (i) the L1, L2 and E7 proteins of the papillomavirus, (ii) gp160, p24, p17, Nef and Tat proteins of the HIV1 virus, (iii) the Tax protein o the HTLV1 or 2 viruses, if need be, detoxicated or a protein being derived therefrom.

In order to induce the immune reaction towards the pathogenic antigenic structure, the polypeptide (a) could comprise the pathogenic protein per se.

Some of the above listed pathogenic proteins have immunotoxic properties, such as for example, the Tat proteins of the HIV1 virus, the E7 protein of the papillomavirus or the Tax protein of the HTLV1 virus, and could therefore not be used per se as polypeptide (a) of a composite superimmunogen.

The polypeptide (a) in that case will be preferably selected amongst (i) an inactivated form, for example detoxicated, of the pathogenic protein, (ii) an inactive fragment of the pathogenic protein and (iii) a protein derived from the pathogenic protein such as a genetic mutant, or a fragment of such a protein, free from the immunotoxic initial properties.

Various techniques for preparing a polypeptide (a) such as herein above defined are explained further in the specification.

Preferably, the polypeptide (a) and the polypeptide (b) are selected amongst the following:

a) For Preventing or Treating AIDS:

polypeptide (a): gp160, p24, p17, nef, or Tat proteins of the HIV1 virus, detoxicated or stabilized if required, immunogenic fragments of such proteins or also an immunogenic protein being derived therefrom (Zagury et al., 1998);

polypeptide (b): Tat, IFNα, and TGFβ proteins, detoxicated if required, immunogenic fragments of such proteins ou an immunogenic protein being derived therefrom;

b) For Preventing or Treating the Neck of the Womb Cancer:

polypeptide (a): L1, L2 and E7 proteins of the papillomavirus, preferably of a papillomavirus from strain 16 or 18, detoxicated or stabilized if required, immunogenic fragments of such proteins or also an immunogenic protein being derived therefrom (Le Buanec et al., 1999);

polypeptide (b): E7, IFNα, TGFβ, TNFα and VEGF proteins, detoxicated or stabilized if required, immunogenic fragments of such proteins or an immunogenic protein being derived therefrom;

c) For Preventing or Treating the ATL Leukaemia Induced y the HTLV1 or 2 Viruses:

polypeptide (a): gp61 and Tax proteins of the HTLV1 or 2 viruses, detoxicated if required, immunogenic fragments of such proteins or also an immunogenic protein being derived therefrom (Cowan et al., 1997; Mori et al., 1996);

polypeptide (b): Tax, IL10, IFNα or TGFβ proteins, detoxicated, immunogenic fragments of such proteins or also a protein being derived therefrom;

d) For Preventing or Treating the Colon Cancer:

polypeptide (a): CEA and p53 proteins, detoxicated if required, immunogenic fragments of such proteins or also an immunogenic protein being derived therefrom (Zusman et al., (1996);

polypeptide (b): TGFβ, IL10, p53, FasL and VEGF proteins, detoxicated, immunogenic peptidic fragments of such proteins or also an immunogenic protein being derived therefrom;

e) For Preventing or Treating Breast Cancer:

polypeptide (a): Di12 protein, immunogenic fragments of such a protein or also a protein being derived therefrom (Yoshiji et al., 1996);

polypeptide (b): TGFβ, TNFα and VEGF proteins, detoxicated if required, immunogenic fragments of such proteins or also an immunogenic protein being derived therefrom;

f) For Preventing or Treating Pancreas Cancer:

polypeptide (a): CaSm protein, detoxicated if this is required, immunogenic fragments of such a protein or also an immunogenic protein being derived therefrom;

polypeptide (b): proteins VEGF and TNFα proteins, detoxicated or stabilized if required, immunogenic fragments of such proteins or also an immunogenic protein being derived therefrom;

g) For Preventing or Treating Prostate Cancer:

polypeptide (a): OSA and ETS2 proteins, detoxicated or stabilized if required, immunogenic fragments of such proteins or also an immunogenic protein being derived therefrom (Sementchenko VI et al., 1998);

polypeptide (b): IL6 and TGFβ proteins, detoxicated or stabilized if required, immunogenic fragments of such proteins or also an immunogenic protein being derived therefrom. (Adler et al., 1999).

For preventing or treating some other cancers, it is also possible to use, as an immunogenic polypeptide (a), TSA antigens (for <<Tumor Specific Antigen type>>) or TM antigens (for <<Tumor Associated Antigen type>>), immunogenic fragments of such proteins or an immunogenic protein being derived therefrom.

Gp160, Tat, Tax and p53 proteins are toxic in their native form. Preparing an immunogenic polypeptide (a) from such proteins will imperatively require to detoxicate them, or to prepare non toxic immunogenic peptidic fragments therefrom or also a non toxic protein being derived therefrom for example by means of one or more substitutions, deletions or additions of amino acids, relative to the reference native protein sequence.

IFNα, Tat, TGFβ, E7, Tax, p53, and FasL proteins, at a high concentration, are immunotoxic, in their native form. Preparing an immunogenic polypeptide (b) from such proteins will imperatively require to detoxicate them, or to prepare non toxic immunogenic peptidic fragments therefrom or also a non toxic protein being derived therefrom for example by means of one or more substitutions, deletions or additions of amino acids, relative to the reference native protein sequence.

The VEGF immunogenic protein could be used, as a polypeptide (b) making up a composite superimmunogen, either under its native form, or under form stabilized by chemical treatment, preferably a chemical treatment of a type used for detoxicting proteins.

The above-listed immunogenic polypeptides (b), and being a cytokine factor or a cell regulating factor with immunotoxic or angiogenic properties, have the features as detailed hereinunder.

TGFβ, protein: TGFβ being a major immunosuppresive cytokin produced by numerous cancer cells;

IL10 protein: IL 10 also being a major immunosuppresive cytokin, as well as FasL (Fas Ligand);

p53 protein: p53 regulation protein abnormally produced by cancer cells could represent an associated tumor antigen (TAA) as been shown in the prior art. When the p53 protein is released by the abnormal cells and builds up in the extracell stroma compartment, it then acts as an immunosuppressive and apotogenic stroma factor (immunotoxic) on the immune cells, and is therefore an immunotoxic stroma circulating protein, according to the invention towards which is sought the induction of a humoral immune reaction by a composite superimmunogen comprising, as a polypeptide (b), detoxicated p53 protein or a protein being derived therefrom and which has lost the immunotoxic properties of the initial protein.

VEGF, growth factor of the endothelial cells: VEGF cytokin being a major cytokin of the angiogenesis, activating the proliferation of endothelial cells;

IL6, I'IFNγ and TNFα, pro-inflammatory cytokins also involved in the ongiogenesis proceses, activating the expression of adherence molecules of the endothelial cells (ICAM, VCAM and E selectin).

The humoral immune reaction being induced by the polypeptide (b) making up a composite superimmunogen according to the invention makes it possible to make efficient the specific immune reaction of the polypeptide (a):

(i) neutralizing or blocking the immunosuppressive activity (the immunotoxicity) of some abnormally produced stroma local circulating proteins, such as IFNα or the Tat protein; or

(ii) neutralizing or blocking the apoptogenic properties towards the immune cells (the immunotoxicity) induced by some abnormally produced stroma circulating proteins, such as the p53 cell regulation factor; or

(iii) neutralizing or blocking the angiogenesis, including the neovascularization of tumors, induced by some abnormally produced stroma circulating proteins, such as VEGF.

A first preferred family of composite superimmunogens useful for preventing or treating some cancers and some viral infections comprises the following composite superimmunogens:

composite superimmunogen (a) gp160—(b) toxoid Tat;

composite superimmunogen (b) Tat peptide [1-15 ;46-60]—(a) gp160;

composite superimmunogen (a) toxoid Tat—(b) IFNα;

composite superimmunogen .(a) toxoid Tat—(b) Tat peptide [1-15 ;46-60]

A second preferred family of composite superimmunogens comprises the following superimmunogens:

composite superimmunogen (a) L1—(b) E7;

composite superimmunogen (a) E7—(b) VEGF;

Herein above, <<(a)>> represents the polypeptide (a) and <<(b)>> represents the polypeptide (b). The structure of the above-listed composite superimmunogens goes, from left to right, from the NH₂-terminal part to the COOH-terminal part.

In the composite superimmunogen (a) gp160—(b) toxoid Tat, gp160 protein induces a cell immune reaction towards VIH1 infected cells, an immune reaction which is made easier by the induction of antibodies neutralizing or blocking the immunotoxic properties of the Tat protein of HIV1, known for generating the production of IFNα, immunosuppressive cytokine factor.

In the (a) L1—(b) E7 superimmunogen, L1 protein of HPV induces a cell immune reaction towards HPV infected cells, which could be cancerous, an immune reaction which is made easier by the induction of antibodies neutralizing or blocking the immunotoxic properties of E7 protein of HPV.

E7 protein, which is expressed by the cancer cell of the neck of the womb cancer, which, therefore, could also be the target of the cytotoxic T cells or of NK cells recognizing the pathogenic antigenic structure, could also be used, for preparing a polypeptide (a) of a composite superimmunogen, either under the form of detoxicated E7 protein or under the form of a protein deriving from E7 protein and which has lost its immunotoxicity.

In the composite superimmunogen (a) E7—(b) VEGF, E7 protein induces a cell immune reaction towards HPV infected cells, which could be cancerous, an immune reaction which is made more efficient because of the induction of antibodies neutralizing or blocking the angiogenic properties of the VEGF cytokine factor.

### Composite Superimmunogens used for Preventing or Treating Allergies

In a composite superimmunogen used for preventing or treating allergies, the polypeptide (a) induces a humoral immune reaction towards the allergenic pathogenic protein making up the particle pathogenic antigenic structure, which could be, by way of an illustration, pollen, an acarid, a dust or a cat hair.

Preferably, the allergenic pathogenic protein is selected from Betv1a, Der p 1 and Fel d 1 proteins, well known to the man of the art.

Preferably, the polypeptide (a) is selected amongst Betv1a, Der p 1 and Fel d 1 proteins, their immunogenic peptidic fragments or also an immunogenic protein being derived therefrom.

Betv1 antigen is disclosed in particular by Ferreira et al. (1993), Der p 1 antigen is disclosed in particular by Tovey et al. (1981) and Fel d 1 antigen is disclosed in particular by Morgenstern et al. (1991)

Preferably, the polypeptide (b), within the composite superimmunogen, induces the production of antibodies neutralizing or blocking towards the IL4 cytokin factor, which is primarily produced by T lymphocytes of Th2 type, orienting the humoral immune response towards the production of IgE isotope antibodies.

The polypeptide (b) is therefore preferably the autologous IL4 protein, preferably detoxicated by chemical, physical or genetic treatment, an immunogenic peptidic fragment of such protein or also a protein derived therefrom.

According to another embodiment, the polypeptide (b) induces the production of antibodies neutralizing or blocking towards the IL5 cytokin factor, which is primarily produced by T lymphocytes T of Th2 type.

According to a second embodiment, the polypeptide (b) is preferably the autologous IL5 protein, preferably detoxicated, an immunogenic peptidic fragement of such protein or also a protein being derived therefrom.

In the composite superimmunogen (a) Betv1a—(b) IL4 (mouse), the Betv1a protein induces a humoral, systemic or mucosal immune reaction, towards the allergenic pathogenic antigenic structure by the production of IgG or IgA isotope antibodies, preferably secretory IgA and the IL4 protein induces an immune reaction towards such a cytokine factor, allowing to inhibit or to block the isotypic switch towards the production of IgE antibodies, at the origin of allergic symptoms, while inducing an efficient immune response towards the causal allergen. The resulting humoral immune reaction towards IL4, which is an autologous protein, is made possible by virtue of the simultaneous presence of the polypeptide (a) also making up the composite superimmunogen, the polypeptide (a) comprising auxiliary T epitopes inducing the activation of auxiliary T cells required for the humoral immune reaction of B cells directed against IL4.

An <<auxiliary T epitope>> is a region of the immunogenic polypeptide which is selectively recognized by the antigen of at least one clone of auxiliary T lymphocytes, the linking event of the immunogenic polypeptide to the receptor of the T lymphocytes specifically recognizing such epitope region activating said T lymphocyte clones, which are going to multiply themselves and to produce cytokins such as IL2, wich in their turn are going to allow the development of a humoral immune reaction, including a humoral immune reaction towards an autologous protein, as is the case for IL4 in a composite superimmunogen according to the invention:

### Inactivation of the Immunosuppressive or Apoptogenic Properties of a Polypeptide (a) or a Polypeptide (b) Making up a Composite Superimmunogen of the Invention, Through Detoxication

As previously explained, a composite superimmunogen used for the invention should be free from any toxicity and should not, more particularly because of the intrinsic properties of one of its components, more specifically the polypeptide (a) or the polypeptide (b), induce any suppression or deviation of the targeted reactions of the immune systems.

Thus, when the polypeptide (a) is an initially immunotoxic polypeptide (a), it is first made inactive for those properties before being included in a composite superimmunogen according to the invention.

Similarly, when a polypeptide (b) making up an immunogenic peptidic conjugate according to the invention is a cytokine or a cell regulation factor with immunotoxic properties, either immunosuppressive or apoptogenic, either able to deviate the immune reaction, for example orienting the reaction of auxiliary T cells (<<T helper>>) towards the production of cells of the Th2 type, as is the case for IL4, it is first made inactive for said properties before being included in said composite superimmunogen.

It is important to use the deleterious polypeptide(s) (a) and/or (b) more particularly under a physically, chemically and/or genetically modified form (inactivated but still immunogenic) and not a native (or natural) form or under an appropriate galenic form such that it no longer exerts the immunotoxic effects or the deviation properties of the immune reaction of the native protein, while retaining its immunogenic properties.

On the contrary, a non immunotoxic polypeptide (a) or a polypeptide (b), such as VEGF, could be used in the native condition or under a modified form after a stabilisation treatment.

The physical treatments could be performed by means of heat, UV radiations, X rays or contact with a O₂ rich atmosphere. Such physical treatments generating intramolecular modifications between chemical moieties (thiol groups for example) can appropriately change the molecule conformation and functionally inactivate it while maintaining its immunogenic properties.

The chemical treatment could occur using a coupling agent such as a dialdehyde, or a carrier protein activated by a pretreatment using a dialdehyde, preferably or glutaraldehyde. The chemical treatment could occur using a monoaldehyde, more particularly formaldehyde. One could refer, on that subject, to the teachings from WO-A-96/27389.

The chemical treatment could occur more particularly using other methods such as carboxymethylation or carboxamidation. An example of a carboxymethylation technique is illustrated in WO-A-99/33872. The chemical treatment could also occur through N ethylmaleidation whether associated or not with a glutaraidehydation.

One could also mention as an inactivation technique the reaction of at least one thiol function of the protein with ammonium 4-chloro-7-sulfobenzofurazan, N-[iodoethyl]-trifluoroacetamid or N-(6-[7-amino-4-methylcoumarin-3-acetamido]hexyl)-3′-(2′-pyridyldithio)-propionamid as well as the reaction of at least one amino function of the protein with ethylacetimidate, an anhydride, 2-iminothiolane hydrochlorate, N-succinimidyl S-acetylthioacetate, sulfosuccinimidyl acetate, sulfosuccinimidyl-4-O-[4,4-dimethoxytrityl]butyrate, succinimidyl 7-amino-4-methylcoumarin-3-acetate, sulfosuccinimidyl 7-amino-4-methylcoumarin-3-acetate or phenylglyoxal.

The immunogen could also be inactivated using a galenic presentation within an oily liquid, such as the Freund's incomplete adjuvant, or also able to modify the non covalent links (electrostatic forces, Van der Waals' forces or hydrogen links) required for its toxic effects.

The genetic modifications could be obtained through genetic engineering performing insertions, deletions or substitutions of residues, operations being designed for reducing or suppressing deleterious functional sites of the natural molecule. The genetic mutants could be subjected or not to a complementary chemical and/or physical treatment. The hereinabove modified proteins could for example be prepared from a protein with a sequence identical or similar to a peptidic sequence of an immunopathogenic protein, in particular immunosuppressive or angiogenic, such as the VIH-1 Tat protein, E7 protein of the papillomavirus or HTLV1 Tax protein or a fragment of such proteins and could be obtained for example through traditional peptidic synthesis on a resin or through genetic engeneering. All such methods are well known in the state of the art. The inactive but immunogenic mutants are at least one DNA molecule coding their production. Such DNA molecules are of particular interest in the present invention as will be seen hereinafter.

In order to check whether the immunotoxic and/or angiogenic native protein is appropriately recognized by the antibodies directed against said modified protein, detoxicated or stabilized by treatment, or its modified or not modified fragment according to the invention, one could for example immunologically check via Elisa in the presence of specific antibodies, the formation of antigen-antibody complexes.

In the preferred implementing conditions, the immunogenic compound originates form a native compound (protein or poypeptidic fragment) treated by an aldehyde, or carboxamide, or carboxymethyl, or maleimide.

In order to determine whether the immunogenic properties of the immunotoxic and/or angiogenic modified protein or of a fragment of such protein were satisfactorily maintained (i.e. whether it has been inactivated but not denaturated) for creating antibodies blocking the effects of said native protein, one could for example immunize mammals (rabbits, rats, mice) using an immunogenic compound according to the invention and check whether the antibodies being produced neutralize the immunosuppressive, apoptogenic or angiogenic activities of the protein.

In order to determine whether the modified immunotoxic protein or the fragment has lost at least the desired proportion of its immunotoxic properties, one could for example study the effect of the modified protein on the production of Th2 (IL4) and Th1 (IFNγ) cytokins and/or on the immunosuppression of cultures mononucleated cells of the human peripherial blood (PBMC) stimulated by booster antigens, such as the PPD or toxoid tetanus antigens.

The modified and angiogenic immunotoxic (immunosuppressive or apoptogenic or leading to a deviation of the immune reaction) or angiogenic protein could derive from any proteins more particularly from local action immunotoxic proteins induced by tumor cells by infected cells of AIDS patients; it is more particularly to be mentioned the Tat protein of the VIH-1 virus, the E7 protein of the papilloma virus or the Tax protein of the HTLV1 virus. It is also to be mentioned the mannan-dependent lectin produced by activated immune cells. It is also to be mentioned the VEGF with angiogenic properties and IL4 with immunotoxic properties and leading to a deviation of the immune reaction towards a Th2 response.

As already explained hereinabove, the polypeptide (a) or the polypeptide (b) of a composite superimmunogen according to the invention could also consist in a fragment of the initial immunogenic protein making up the targeted pathogenic antigenic structure, or also a polypeptide having, relative to the initial immunogenic protein, modifications in the amino acids.

It could comprise one or more modifications in the amino acids of such a protein or fragment such as deletions, substitutions, additions, or functionnalizations such as amino acid acylation, with the proviso that such modifications should remain in the scope as hereinabove defined (lack of toxicity, immunologic features). For example, generally, replacing a leucin residue by an isoleucin residue does not modify such properties; the modifications should generally apply to less than 40% of the amino acids, preferably less than 20% and more preferably less than 10% of the immunopathogenic protein, in particular immunotoxic or angiogenic. It is important that the modified protein or fragment should not be denatured as this could occur for example using such a physical treatment as heat in order to maintain its conformation sites so that antibodies induced by modified derivates would be active towards the native protein.

In preferred conditions, the immunogenic compounds of the invention comprise at least 50% of the whole or a segment of the immunopathogenic protein, in particularl immunotoxic or angiogenic, preferably at least 70%, more preferably at least 90%, and more particularly all or nearly all said immunosuppressive or angiogenic protein.

Generally, as far as the modifications are concerned, the homology or the similarity between the modified immunogen and the protein or part of the native immunotoxic protein, as well as the dimensions of the immunogenic polypeptide, as well as the use or the coupling conditions of the immunogenic polypeptide making up a composite superimmunogen according to the invention to an immunogenic protein such as the tetanic toxoid, one could more particularly see WO-A-86/06 414 or EP-A-0.220.273 as well as PCT/US 86/00831, being equivalent, their teachings being incorporated herein by reference.

Is also preferred an immunogenic compound such as defined herein above which is a product obtained through genetic recombination having a peptidic homology of at least 70% with the Tat protein of HIV-1, the Tax protein of HTLV1 or L2 and the E7 protein of HPV or the mannan-dependent lectin produced by activated immune cells or a segment of such proteins.

Is also preferred an immunogenic compound such as herein above defined characterized in that it is treated by an aldehyde, whether carboxamide, carboxymethyl or maleimide.

Is further preferred an immunogenic compound such as hereinabove defined characterized by an adjuvant conditioning making it biologically inactive, such as an oily emulsion into a Freund's incomplete adjuvant (IFA).

One could also derive the desired immunogenic compound from a homologous mutant.

It should be noted here that using a galenic conditioning of a physiologically active protein, one could hide its biological activity while maintaining its immunogenicity.

The carboxymethylation reaction makes it possible to modify the thiol groups (sulfhydryl groups) present at the level of the cysteine residues of the amino acid sequence. The carboxymethylation, a technique well known to the man of the art, inactivates some toxic functions depending on the SH groups according to the technique disclosed for the Tat protein by Frankel et al. (Cell, vol. 55 1988).

Except for the carboxymethylation, the carboxamidation or the maleimidation could be used for blocking the SH groups and forming S-carboxyethyle, S-carboxamide or S-maleimide complexes.

For example, the Tat protein has 7 cysteins. Such systeins are involved in forming inter- and intra-chain disulfide bridges and contribute to forming oligomers.

The reaction product is in each case a S-carboxymethylcysteinyl or a S-carboxymethylamidocysteinyl residue.

A fragment could comprise 8 to 110 amino acids for example, preferably from 12 to 60 amino acids and more particularly from 12 to 40 amino acids. Such a fragment could also comprise or or more C or N terminal sides of 1 to 5 additional amino acids i.e. different from the original fragment. A fragment should additionally comprise a cysteine at least for being subjected to, for example, a carboxymethylation. The fragments, if they are preferably selected inactivated per se, could indeed be subjected, if wanted, to the same inactivation treatment as the entire or nearly entire proteins.

The above-mentioned carboxymethylation reaction could as well occur with other chemical agents such as performic acid, 3-bromopropionic acid, ethyleneimine, (2-bromoethyl)trimethylammonium bromide, 2-bromoethane sulfonate,1,3-propanesulfone etc.

In the preferred implementing conditions of the above-described method, said starting protein or said starting fragment may have a fused form with a marker (FP) or a non fused form (P). The FP form could modify per se the molecular conformation and hence, modify its activity.

The starting proteins or fragments of the method are known products, their inactivation methods being described in the literature as in WO-a-99/33872. Such starting proteins could even be commercialized (immunodiagnostics Inc., Cat # 1002-2) or could be traditionally prepared.

One could in particular prepare the above-mentioned starting proteins or fragments through:

1) synthesis by genetic engineering or by biochemical synthesis;

2) purification.

Using genetic engineering, one could purify the produced proteins through affinity chromatography using for example antibodies raised against the protein or one of its fragments; one could also synthesize the protein being fused with a marker (FP) which will serve for the attachment to an affinity column.

### Preferred Embodiments of a Composite Superimmunogen of the Invention

In order to produce a composite superimmunogen according to the invention, a coupling is made of the polypeptide (a) and of the polypeptide (b) through a chemical route or a genetic recombination.

According to a preferred embodiment of the peptidic conjugate of the invention, the polypeptides (a) and (b) are inactivated by their immunotoxic properties and/or stabilized through formolation, carboxamidation, carboxymethylation, maleimidation or oxidation with oxygen bubbling.

In another aspect, the polypeptides (a) and (b), when they are inactivated in their immunotoxic or angiogenic properties, are inactivated through genetic recombination.

In a particular embodiment of the immunogenic peptidic conjugate of the invention, the polypeptides (a) and (b) are directly covalently linked between one another, for example via a —CO—NH— peptidic link.

However, in order to introduce some flexibility in the structure of the immunogenic peptidic conjugate, and more particularly allow for some mobility in the space of the polypeptides (a) and (b), one relative to the other within the immunogenic peptidic conjugate, a peptidic conjugate is preferred wherein the polypeptides (a) and (b) are separated from one another, within said conjugate, by a spacer chain.

According to a first preferred embodiment of an immunogenic peptidic conjugate, the polypeptides (a) and (b) are separated from one another, within said conjugate, by a spacer chain selected amongst SMCC or SIAB, which are both bifunctional compounds.

The SIAB compound, as disclosed by Hermanson G. T. (1996, Bioconjugate techniques, San Diego: Academic Press, pp 239-242), is the compound represented by the following formula (I):

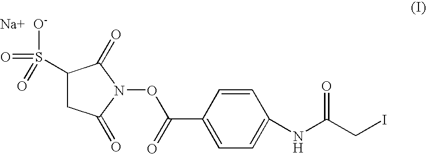

The SIAB compound comprises two reactive groups, respectively an iodoacetate group and an ester sulfo-NHS group, both groups respectively reacting on amino and sulfhydryl groups.

The SMCC compound, which is disclosed by Samoszuk M. K. et al. (1989, Antibody, Immunoconjugates Radiopharm., 2(1): 37-46), is the compound represented by the following formula (II):

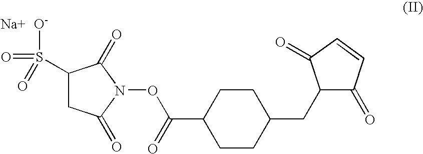

The SMCC compound comprises two reactive groups, respectively a ester sulfo-NHS group and a maleimide group, respectively reacting with an amino group and a sulfhydryl group.

According to a second preferred embodiment, the composite superimmunogen comprises a spacer chain consisting in a linear spacer peptide. A linear spacer peptide will be preferably selected being 3 to 30 amino acids long, advantageously 5 to 20 amino acids long and most preferably 7 to 15 amino acids long.

Preferably, the linear spacer peptide is essentially, namely exclusively, made of positively or negatively charged amino acids at pH 7.0 so as to increase the overall hydrophilicity of said composite superimmunogen. It should be understood that it should be avoided to use spacer peptides consisted of hydrophobic amino acids. Preferably, the spacer peptide is characterized in that it consisted of a poly(lysine) chain made of 3 to 30 lysine residues, more preferably 5 to 20 and most preferably 7 to 15 lysine residues long.

According to another embodiment of a composite superimmunogen according to the invention, the polypeptides (a) and (b) are separated from one another, within said peptidic conjugate, by a spacer chain made of a branched spacer peptide, preferably a oligodendrimeric structure of poly(lysine), such as disclosed for example by Basak et al. (1995).

In the latter embodiment of a composite superimmunogen according to the invention, said peptidic conjugate could comprise several copies of the polypeptides (a) and (b) per conjugate molecule, advantageously 2 to 8 copies of the polypeptides (a) and (b), preferably at the most 4 copies of each of those polypeptides (a) and (b) per conjugate molecule.

The polypeptides (a) and (b) could also be both included within one single physical structure for example on monoparticles having a diameter ranging between 10 and 500 nanometers preferably from 10 to 1000 nanometers, and most preferably between 10 and 100 nanometers, for example IMS nanoparticles, such as disclosed for example by Aucouturier et al. (2001), of chitosan, as disclosed for example by Sjaugrud et al. (1999), liposomes, or biodegradable particles such as acidic polylactide (PLA), poly-ε-caprolactone (PLC) or poly(lactide-coglycolide) (PLG) disclosed by Baras et al. (1999).

According to yet another embodiment, the polypeptides (a) and (b) are physically linked within one single carrier structure allowing for their simultaneous presentation to the cells of the immune system. In such a particular embodiment, the polypeptides (a) and (b) are immobilized on nanoparticles, for example chitosan nanoparticules, IMS nanoparticles, (Immunomodulator available from Societe Seppic Corporation, France), made of lipidic nanoparticles with a diameter ranging from 100 to 300 nanometers) or liposomes.

Preferably, such nanoparticules have a small size so as to simultaneously present the polypeptides (a) and (b) to the cells, as though such polypeptides would be covalently linked with one single molecule. Advantageously the nanoparticles have a diameter ranging between 10 and 1000 nm, preferably between 10 and 500 nm, more prefereably between 10 and 300 nm and most preferably between 10 and 200 nm.

Using an immunogenic peptidic conjugate such as hereinafter defined has a major application for treating AIDS, cancers and allergy.

Another object of the present invention is a composite superimmunogenic compound comprising two distinct immunogenic polypeptides physically linked to one another, both polypeptides respectively consisting in:

(a) a first immunogenic polypeptide inducing a cell immune reaction or a cell and humoral immune reaction towards an inert or living cell, microbial or particle pathogenic antigenic structure;

(b) a second immunogenic polypeptide inducing the production of antibodies neutralizing or blocking towards a stroma local circulating protein selected amongst a cytokin factor or a cell regulation factor with immunotoxic or angiogenic properties, such a factor being either produced by cancer cells, virus infected cells or stroma cells, including T lymphocytes and the cells having the antigen (APC), or induced by pathogenic particle structures, more particularly allergenic.

The detailed features of the above described composite superimmunogen have already been set forth previously in the present specification, to which the man of the art will refer.

In a first preferred embodiment, the immunogenic peptidic conjugate is characterized in that it consists in the gp160—toxoide Tat conjugate.

According to a second preferred embodiment, the peptidic conjugate is characterized in that it consists in the toxoid Tat-IFNα conjugate.

According to a third preferred embodiment, the immunogenic peptidic conjugate is characterized in that it consists in E7-SIAB-VEGF.

According to a fourth preferred embodiment, the immunogenic peptidic conjugate is characterized in that it consists in BetV1a-SIAB-IL-4.

### Use of a Nucleic Acid Coding a Composite Superimmunogen According to the Invention

Another object of the invention is a nucleic acid coding some composite superimmunogenic compounds according to the invention, i.e. peptidic superimmunogens exclusively consisting in one single linear peptidic chain comprising the polypeptides (a) and (b) linked to one another, either directly or via a linear peptidic spacer chain, such as hereinabove defined

It is also relative to a functional expression cassette in a mammal, preferably in man, comprising a nucleic acid such as hereinabove defined, being put under the control of a functional regulating polynucleotide in a mammal, preferably in man.

It also relates to a recombinant vector comprising a nucleic acid or an expression cassette such as hereinabove defined, allowing for the expression of a composite superimmunogen according to the invention in a mammal, preferably in man.

It also relates to using a nucleic acid, an expression cassette or a recombinant vector such as hereinabove defined for obtaining a drug with an anti-cancer, anti-viral or anti-allergic action.

Generally, the DNA (plasmid with a promotor) could be delivered to the mucosal surfaces in the form of bare DNA or formulated, for example, in the form of cationic liposomes or concentrated around gold particles or also under the form of microspheres. It is advantageously implemented in the presence of adjuvants, more particularly, bacterial toxins, such as CTB (cholera toxin) or LT (E. coli labile enterotoxin), preferably mutated (LTμ). Such mucosal immuneization techniques with DNA molecule based vaccines are disclosed in particular in Microbes and Infection, 1999, 685-698 by McCluskie et al.

According to an advantageous embodiment, a recombinant vector according to the invention would more particularly comprise the following elements:

(1) regulation elements for the expression of the nucleic acid coding the composite superimmuneigen such as promoters and enhancing sequences (“enhancers”);

(2) the coding sequence included in the nucleic acid coding a composite superimmunogen to be inserted into such a vector, said coding sequence being put in phase with regulation signals such as described in (1); and

(3) appropriate initiation and stopper sequences of the transcription.

In addition, the recombinant vectors according the invention could comprise one or more replication origins in the cell hosts for which their amplification or their expression is being sought, markers or selection markers.

A recombinant vector according to the invention could also be a retroviral vector or also a DNA associated vector (AAV). Such DNA associated vectors are for example disclosed by Flotte et al. (1992), Samulski et al. (1989), as well as McLaughlin B A et al. (1996).

In order to introduce polynucleotides or vectors into a host cell, the man of the art could advantageously use various techniques, such as the calcium phosphate precipitation technique (Graham et al., 1973; Chen et al., 1987), the Dextran DEAE (Gopal, 1985), the electroporation (Tur-Kaspa, 1896; Potter et al., 1984), the direct microinjection (Harland et al., 1985), the DNA loaded liposomes (Nicolau et al., 1982, Fraley et al., 1979).

According to a particular embodiment, a method for introducing a polynucleotide according to the invention into a host cell, in particular a host cell from a mammal, in vivo, comprises a step wherein a preparation is introduced, said preparation comprising a pharmaceutically compatible vector and a “bare” polynucleotide according to the invention, under the control of appropriate regulation sequences, through local injection at the level of the selected tissue, for exemple a smooth muscle tissue, the “bare” polynucleotide being absorbed by cells of such a tissue.

Compositions for the in vitro and in vivo uses comprising “bare” polynucleotides are for example disclosed in the PCT application N^{o} WO 95/11307 (Institut Pasteur, Inserm, Ottawa University) as well as in the articles by Tacson et al. (1996) and by Huygen et al. (1996).

They could also be adenoviral vectors such as the type 2 or 5 human adenovirus.

The vector amount being injected into the selected host organism varies depending on the injection site. By way of indication, there could be injected from approximately 10 to 1,000 μm of the polynucleotide coding a composite superimmunogen in the body of an animal, preferably of a patient.

### Immunogenic or Vaccinial Compositions Comprising Either a Composite Superimmunogen or a Nucleic Acid Coding Same

Another object of the invention is an immunogenic composition characterized in that it comprises, as an active, a composite superimmunogen such as herein above defined, in association with one or more physiologically compatible excipients.

It relates to an immunogenic composition characterized in that it comprises an efficient amount, for example immunologically active, of a composite superimmunogen such as defined in the present specification.

Depending on the objects being sought, systemic adjuvants or mucosal adjuvants are used. For example, preferably a mucosal adjuvant is used for preventing cancers of the epithelial tissues and preferably systemic adjuvatns are used for preventing or treating virus infections, such as HIV1 and HTLV1 virus infections, or also for preventing or treating allergies.

Amongst the systemic adjuvants, are preferably used type IFA (Freund's Incomplete Adjuvant) adjuvants, calcium phosphate or alumina hydroxide.

Amongst the mucosal adjuvants are preferably used adjuvants such as chloratoxin B (CTB) or a mutant of toxin LT (LTμ).

It is also relative to a mucosal or systemic vaccine, characterized in that it comprises, as an active, a composite superimmunogen such as hereinabove defined in association with one or more physiologically compatible excipients, including immunity adjuvants,

The immunogenic compositions or vaccines according to the present invention are useful for example in both curative and cancer treatment, including virus induced cancers such as for example, ATL (Acute T cell leukaemia) caused by HTLV1 or neck of the womb cancer caused by the papilloma virus, or also the Burkitt's lymphoma or the Kaposi's sarcoma caused by viruses of the Herpes family as well as in the treatment of AIDS as well as for preventing or treating allergic inflammatory reactions.

The composite superimmunogens according to the invention could be used as follows:

A patient is administered, under a form adapted for the systemic or mucosal administration, a composite superimmunogenic compound or a DNA molecule according to the present invention, for example, intranasally, in an amount sufficient for being therapeutically efficient, to an individual needing such a treatment. The dosage to be administered could range for example from 10 to 1,000 μg intranasally once a week for two months, then periodically depending on the induced secretory antibody rate, for example, every 2-6 months.

Two or more different composite superimmunogenic molecules and/or DNA molecules could be administered in one single preparation for inducing antibodies neutralizing all the deleterious functional sites in the case where one single molecule does not carry all the active sites of the overproduced toxin or the cytokine that is to neutralized.

Another object of the invention is also to provide pharmaceutical compositions designed for the mucous membranes containing at least one above-mentioned immunogenic peptidic conjugate or DNA molecule as an active.

When used as drugs, the immunogenic peptidic conjugates or the DNA molecules of the invention could be incorporated into pharmaceutical compositions designed for a systemic administration or for a mucosal administration, including oro-mucosal, in particular intranasal, oral and vaginal. The administration could occur in a single dose or could be repeated once or several times after some time interval.

That is why the present application also relates to a curative or preventive pharmaceutical composition, characterized in that it comprises as an active, one or more composite superimmunogens such as hereinabove defined, or their fragments or DNA molecules corresponding to the native protein to be overcome. The immunogenic compound, DNA fragment or molecule could be conditioned alone or blended with one excipient or blend of pharmeutically acceptable excipient blends such as an adjuvant. Amongst the excipients designed for the intranasal or oral route, are particularly worth mentioning capryl caproyl macrogol glycerides such as Labrasol® from GATTEFOSSE Corporation or alumina hydroxide (Alhydragel, Superfos, Denmark).

It should be noted that when administered as such, according to a traditional oral formulation, the active according to the invention would be inactive.

For the oral administration according to the invention, the active would be associated with a mucosal immunity adjuvant such as a CT, LT or CTB mutant.

Are to be particularly mentioned the galenic forms as disclosed by Boyaka et al. <<Strategies for mucosal vaccine development>> in Am. J. Trop. Med. Hyg. 60(4), 1999, pages 3545. Are also to be mentioned the gastroresistant. microgranules, including bioadhesives such as those disclosed by Rojas et al. in Pharmaceutical Research, vol. 16, no. 2, 1999, page 255.

In the particular implementation conditions, there is to be mentioned an above-mentioned vaccinial pharmaceutical composition, characterized in that it comprises a mucosal immunity adjuvant, such as a CT mutant (cholera toxin) or a LT mutant (E. coli labile enterotoxin).

In other particular implementing conditions, there is to be mentioned an above-mentioned vaccinial pharmaceutical composition, characterized in that it comprises an adjuvant absorbing the active principle, such as alumina hydroxide or gold particles

In yet other preferred implementing conditions, there is to be mentioned an above-mentioned pharmaceutical composition, characterized in that the immunogenic peptidic conjugate is obtained through genetic recombination.

In still other preferred implementing conditions, there is to be mentioned an above-mentioned pharmaceutical composition, characterized in that the polypeptide (a) and/or the polypeptide (b) making up the peptidic conjugate has been aldehyde treated, carboxymethylated, carboxamidated or maleimidated.

Still another object of the present invention is a method for preparing an above described composition, characterized in that the active(s) is or are mixed, using methods known per se, with acceptable excipients, and, if need be, with a systemic or mucosal immunity adjuvant.

In preferred implementing conditions of the above-mentioned method, bioadhesive and gastroresistant microgranules are prepared for the oral digestive route comprising the immunogenic actives and, if need be, the adjuvants.

Another object of the invention is an immunogenic composition characterized in that it comprises a therapeutically efficient amount of a nucleic acid coding a composite superimmunogen such as defined in the present specification, an expression cassette comprising such a nucleic acid or a recombinant vector comprising such a nucleic acid or such an expression cassette.

It also relates to a vaccine characterized in that it comprises a therapeutically active amount of a nucleic acid, an expression cassette or a recombinant vector such as hereinabove defined

The present invention is additionally illustrated by the following examples.

### EXAMPLES

### Examples of Composite Superimmunogen Preparations

### Example 1

### Preparation of a [gp160—Toxoid Tat] Conjugate

Such a conjugate should represent the active of a composite vaccine able to primarily induce in the vaccine a cell reaction (chemiokins; auxiliary T, CTL) raised against the infected cells expressing the gp160 and an antibody reaction against the extracell Tat protein.

810 μg of gp160 protein dissolved in 1 ml PBS were activated through dialysis, overnight, against 100 ml of a 0.2% glutaraldehyde solution in PBS, thereafter, the glutaraldehyde excess has been eliminated by 3 successive dialyses against 100 ml PBS, of 2 hours each.

To so activated 1 ml gp160 were added 522 μl of a Toxoid Tat solution at 1.55 ml/ml (i.e. 810 μg of toxoid Tat). The mixture has been stirred overnight, at 4° C., and then the reaction has been blocked through the addition of 50 μl of a 2.5 M glycine solution. The reaction mixture has been finally purified through exclusion chromatography. The antigenicity of the preparation towards Tat protein and gp160 protein corresponds to the antigenicity of each of the individually determined proteins

### Example 2

### Preparation of a [Toxoid Tat—IFNα] Conjugate

This involves the production of a composite vaccine able to induce primarily a cell immune reaction (chemiokins; auxiliary T, CTL) raised against infected cells expressing the Tat protein and a humoral immune reaction being raised against IFNα. In addition such a conjugate will be able to induce the formation of antibodies raised against the extracell Tat protein.

Coupling occurs through reaction of IFNα reduced with the toxoid Tat molecule activated by SIAB (cf. ex. 2).

1. Activation of the toxoid Tat (Tx)

5 mg of Tx dissolved in 3 ml of 5mM PBS-EDTA 5mM have been added with 187 μl of a SIAB solution (1.7 mg/ml). After one hour of reaction at laboratory temperature, the reaction mixture has been filtered through a Sephadex G25 column equilibrated in 0.1 M-EDTA 5 mM borate buffer, pH 8.5.

2. Reduction of IFNα

The conditions are the same as in example 3. Recovery of 2.6 mg of reduced IFNα.

5 mg of Tx-SIAB have been mixed under stirring with 2.6 mg of reduced IFNα and stirring has been continued at laboratory temperature for 1 hour and then overnight at 4° C. The reaction has been blocked through the addition of Cys, HCl at a 5 mM final concentration and reaction for 30 min.

The reaction mixture has been purified through exclusion chromatography. The antigenicity of the toxoid-Tat and of IFNα has been found to be maintained without the conjugate.

### Example 3

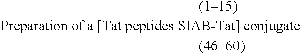

This involves the production of a composite vaccine able to primarily induce a cell immune reaction (chemiokins; auxiliary T, CTL) raised against infected cells expressing Tat protein and a humoral immune reaction raised against the extracell Tat protein.

1. Activation of the Tat Peptides with SIAB

A 1.5 mg mixture of each of the peptides (1-15 and 46-60) dissolved in 600 μl of water has been activated through treatment, for 1 hour, at laboratory temperature with 500 μl of a SIAB solution at 1.7 mg/ml in PBS, then the reaction mixture has been filtered on Sephadex G25 (1×15 cm column) equilibrated in PBS buffer.

2. Reduction of the Tat Protein

Tat reduced by 2 mercaptoethylamine (cf. conditions in example 3).

3. Coupling of the Tat Protein to the Tat Peptides

0.75 mg of Tat peptides activated by SIAB have been mixed with 1.25 mg of reduced Tat, and the mixture has been stirred for 45 min. at laboratory temperature, then stored overnight at 4° C. The reaction has been blocked through the addition of Cys, HCl at a 5 mM final concentration, then the mixture has been purified, filtration through calibrated membranes. The Tat protein in the conjugate has been inactivated through carboxamidation.

The antigenicity of the peptide Tat-toxoid Tat conjugate compared to that of the toxoid Tat has been determined using a traditional indirect ELISA. The peptide Tat-toxoid Tat conjugate has antigenicity equal to the antigenicity of the toxoid Tat protein.

### Example 4

### Preparation of a [E7-SIAB-VEGF) Conjugate

Such a conjugate represents the active of a composite vaccine able to primarily induce in the vaccine a cell reaction (chemiokins; auxiliary T, CTL) raised against infected cells expressing the E7 protein and an antibody reaction against the VEGF protein. In addition, such a conjugate will be able to induce the formation of antibodies raised against the extracell E7 protein.

1. Activation of the E7 Protein

1 mg of E7 protein is dissolved in 1 ml of 0.1 M—EDTA 5 mM borate buffer, pH 8.5, containing 20% of dioxane. To that solution are added 400 μl of a SIAB solution at 1.7 mg/ml dissolved in the same buffer. The reaction continues for 1 hour at laboratory temperature and the reaction mixture is applied to a Sephadex G25 column (1×16 cm) equilibrated with the borate-EDTA-dioxane buffer and fractions corresponding to the protein are recovered and pooled.

2. Reduction of the VEGF Protein

The reduction occurs through reaction with 2-mercaptoethylamine in the conditions as described in example 3.

3. Coupling Reduced VEGF to E7-SIAB

1 mg of reduced VEGF is allowed to react with 1 mg of E7 activated by sulfo-SIAB. After 1 hour of reaction at laboratory temperature, the reaction has been blocked through the addition of cysteine at a 5 mM final concentration and the reaction mixture concentrated through diffusion against solid saccharose. The concentrated solution has been finally purified through exclusion chromatography. The antigenicity of E7 and of VEGF is maintained in the conjugate.

### Example 5

### Preparation of a [Bet V 1a—SIAB—II-4 (murine)] Conjugate

Such a conjugate represents the active of a composite vaccine designed for orienting the response towards the formation of IgG class antibodies and not IgE anymore against the Bet V 1a birch-tree allergen.

1. Reduction of II-4

1 mg of II-4 dissolved in 250 μl of 10 mM phosphate buffer, pH 7.2+1 mM EDTA has been reduced by 2-mercaptoethylamine according to the conditions in example 3 and the reduced cytokine recovered through gelation on Sephadex G25.

2. Activation of the Bet V 1a Protein by SIAB

1 mg of Bet V 1a protein dissolved in 500 μl of 10 mM—EDTA 1 mM phosphate buffer has been diluted by 80 μl of a solution at 1.7 mg/ml of sulfo-SIAB for 2 hours at laboratory temperature. The reaction mixture has been purified through exclusion chromatography.

3. Coupling of the Reduced IL4 to Bet V 1a-SIAB

1 mg of reduced IL4 has been allowed to react with 0.5 mg of Bet V 1a activated by sulfo-SIAB. After 1 hour of reaction at laboratory temperature, the reaction has been blocked through the addition of cysteine at a 5 mM final concentration and the reaction mixture concentrated through diffusion against solid saccharose. The concentrated solution has been finally purified through exclusion chromatography. The antigenicity of IL4 and of Bet V 1a is maintained in the conjugate.

### Examples of Immunogenic Activity of Superimmunogens

### Example 6

### Immunogenic Activity of the gp160-toxoid Tat Conjugate

A. Material and Methods

The immunogenic activity of the gp160-toxoid Tat preparation compared to that of toxoid Tat alone and its lack of toxicity have been determined in 18-20 g Balb c mouse.

1-Immunization:

At day 0, a group of 3 mice receive a 0.2 ml (50 μg) injection of an ACF emulsion through intramuscular route. A 5 μg boosting injection in AIF is given at day 21 and day 60.

A blood sample at the retro-orbital level is taken from each mouse before the first injection at day 2.

3 control mice receive the same preparations without immunogen.

The mice are sacrificed 12 days after the last immunization.

2-Toxicity:

The abnormal toxicity is studied in 3 mice receiving 1 human dose (100 μg) according to the pharmacopeia.

The lack of immunotoxicity of the superimmunogen is evaluted in vitro using a cell proliferation test conducted on PBMCs cultivated in the presence of the conjugate and stimulated by PPD or toxoid Tetanos.

B. Results:

1-Lack of Toxicity of the Superimmunogen in vivo and in vitro

The mice immunized as well as by the gp160-toxoid Tat preparation as by the toxoid Tat alone do not show any clinical sign and no anatomic wound. The immunosuppression test shows that the 100 ng/ml doses at 3 μg/ml of gp160-toxoid Tat do not decrease the lymphocyte proliferation.

None of the 3 mice immuneized with 100 μg of the preparation does show any toxicity signs (temperature, cutaneous disorders, systemic or regional signs) for the 7 days following the injection.

2—Humoral Response

The humoral response is measured by the presence in the serum of IgG type antibodies raised against the native Tat recombinant protein, as measured by ELISA and expressed in titration (opposite of the dilution resulting in an optical density higher than 0.3).

TABLE 1

Titration

| | |
|---|---|
| d-2 | D72 |

control mouse:

| | | |
|---|---|---|
| control mouse 1 | <500⁻¹ | <500⁻¹ |
| control mouse 2 | <500⁻¹ | <500⁻¹ |
| control mouse 3 | <500⁻¹ | <500⁻¹ |

mouse immunized with the toxoid Tat:

| | | |
|---|---|---|
| mouse 4 | <500⁻¹ | 32,000⁻¹ |
| mouse 5 | <500⁻¹ | 48,000⁻¹ |
| mouse 6 | <500⁻¹ | 48,000⁻¹ |

mouse immunized with the gp160-toxoid Tat conjugate:

| | | |
|---|---|---|
| mouse 7 | <500⁻¹ | 64,000⁻¹ |
| mouse 8 | <500⁻¹ | >64,000⁻¹ |
| mouse 9 | <500⁻¹ | >64,000⁻¹ |

The mice immunized with the gp160-toxoid Tat preparation have antibody titrations of the IgG anti-Tat type higher than those of the mice immunized with the toxoid Tat alone.

The neutralizing activity of such antibodies has been measured via the Cat assay. Various serum dilutions (1/50-1/400) taken at d-2 and d-72 are incubated for 2 hours with 50 ng/ml of native Tat. Such dilutions are then put again on HeLa cells, cells stably transfected with a plasmid containing the LTR of the VIH-1 as a promoter of the Chloramphenicol gene Acetyl transferase (CAT). After 24 hours of culture, the cells are lysed and the amount of CAT protein being produced is measured via an ELISA test, the Cat assay (Boehringer Mannheim). The neutralizing serums prevent the Tat protein from inducing the expression of the CAT protein, whereas the non neutralizing serums allow for the synthesis of such a CAT protein. The results are given in neutralization %.

TABLE 2
Mouse immunized with the toxoid Tat:

| | | 1/50 | 1/100 | 1/200 | 1/400 |
|---|---|---|---|---|---|
| mouse 4 | d-2 | 0 | 0 | 0 | 0 |
| | d72 | 75 | 50 | 25 | 20 |
| mouse 5 | d-2 | 0 | 0 | 0 | 0 |
| | d72 | 75 | 60 | 30 | 20 |
| mouse 6 | d-2 | 0 | 0 | 0 | 0 |
| | d72 | 75 | 60 | 30 | 20 |

TABLE 3
Mouse immunized with the gp160-toxoid Tat conjugate:

| | | 1/50 | 1/100 | 1/200 | 1/400 |
|---|---|---|---|---|---|
| mouse 7 | d-2 | 0 | 0 | 0 | 0 |
| | d72 | 100 | 100 | 100 | 75 |
| mouse 8 | d-2 | 0 | 0 | 0 | 0 |
| | d72 | 100 | 100 | 100 | 100 |
| mouse 9 | d-2 | 0 | 0 | 0 | 0 |
| | d72 | 100 | 100 | 100 | 100 |

The antibodies induced by the gp160-toxoid Tat conjugate have a higher neutralizing power than that induced by the toxoid Tat.

3. Cell Response

3.1 Production of MP1α and MP1β in the Culture Supernatants of the Splenocytes

The splenocytes of the immunized mice and the control mice are isolated and then cultivated in round-bottomed wells of a micro-culture plate at a rate of 100,000 cells/well in the presence of 5 μg/ml of p24, gp160, of native Tat and a mixture of 5 μg/ml gp160 and 5 μg/ml native Tat. The supernatants are taken after 24 hours of culture and the presence of MIP1α and MIP1β in the supernatants is measured via an ELISA test of R&D. The results are expressed in pg/ml.

TABLE 4
Control mouse:

| | | | Gp | native | gp160 + | |
|---|---|---|---|---|---|---|
| | | | 160 | Tat | native Tat | p24 |
| mouse 1 | d72 | MIP1α | 110 | 100 | 150 | 10 |
| | | MIP1b | 100 | 90 | 140 | 7 |
| Mouse 2 | d72 | MIP1α | 120 | 95 | 153 | 8 |
| | | MIP1β | 112 | 80 | 114 | 6 |
| mouse 3 | d72 | MIP1α | 124 | 98 | 128 | 9 |
| | | MIP1β | 99 | 112 | 117 | 7 |

TABLE 5
Mice immuneizd by the toxoid Tat:

| | | | | | | |
|---|---|---|---|---|---|---|
| Mouse 4 | d72 | MIP1α | 152 | 122 | 203 | 10 |
| | | MIP1β | 140 | 118 | 196 | 11 |
| Mouse 5 | d72 | MIP1α | 145 | 150 | 215 | 8.5 |
| | | MIP1β | 130 | 160 | 230 | 9 |
| Mouse 6 | d72 | MIP1α | 147 | 222 | 290 | 7 |
| | | MIP1β | 152 | 218 | 300 | 9 |

TABLE 6
Mice immunized by the gp160-toxoid Tat conjugate:

| | | | | | | |
|---|---|---|---|---|---|---|
| Mouse 7 | d72 | MIP1α | 810 | 729 | 1,600 | 7.5 |
| | | MIP1β | 1,100 | 850 | 2,000 | 10 |
| Mouse 8 | d72 | MIP1α | 1,000 | 821 | 1,700 | 8 |
| | | MIP1β | 1,125 | 876 | 2,100 | 9 |
| Mouse 9 | d72 | MIP1α | 975 | 768 | 1,685 | 8 |
| | | MIP1β | 1,000 | 803 | 1,862 | 9 |

The splenocytes of the mice immunized with the gp160-toxoid Tat conjugate produce more MIP1α and MIP1β chemiokins than the cells of mice immunized by the toxoid Tat alone when they are activated, in vitro, by the immunogens used during the immunization.

3.2 Production of Gamma IFN in the Culture Supernatants of the Splenocytes

The presence of gamma IFN in the culture supernatants of splenocytes cultivated in the presence of 5 μg/l of p24, of gp160, of native Tat and of a mixture of 5 μg/ml of gp160 and 5 pg/mi of native Tat is determined after 72 hours of culture via an ELISA of R&D. The results are expressed in pg/ml.

TABLE 7
Control mice:

| | | Gp160 | native Tat | gp160 + nativeTat | p24 |
|---|---|---|---|---|---|
| Mouse 1 | d72 | 0 | 0 | 0 | 0 |
| Mouse 2 | d72 | 0 | 0 | 0 | 0 |
| Mouse 3 | d72 | 0 | 0 | 0 | 0 |

TABLE 8
Mice immunized with the toxoid Tat:

| | | | | | |
|---|---|---|---|---|---|
| Mouse 4 | d72 | ND | ND | ND | ND |
| Mouse 5 | d72 | ND | ND | ND | ND |
| Mouse 6 | d72 | ND | ND | ND | ND |

TABLE 9
Mice immunized with the gp160-toxoid Tat conjugate:

| | | | | |
|---|---|---|---|---|
| Mouse 7 | d72 | 40 | 600 | 6,000 |
| Mouse 8 | d72 | 50 | 620 | 5,950 |
| Mouse 9 | d72 | 65 | 700 | 6,850 |

The splenocytes of the mice immunized with the gp160-toxoid Tat conjugate produce a high amount of gamma IFN gamma when they are activated, in vitro, with the immunogens used during the iimmunization.

3.3. Proliferation of the Splenocytes of Immunized Mice (CMI)

The splenocytes of the immunized mice and the control mice are isolated and then cultivated in round-bottomed wells of a micro-culture plate at a rate of 100,000 cells/well in the presence of p24, gp160, native Tat and a mixture of gp160 and native Tat. The cell culture is continued at 37° C. in a humid atmosphere loaded at 55% of CO2 for 6 days. 18 hours before the end of the incubation, 0.5 μCi of tritiated thymidine/well are added. The intensity of the immune response is proportional to the proliferation index Ip.

TABLE 10
Control mouse:

| | | | | gp160 + | |
|---|---|---|---|---|---|
| | | Gp160 | native Tat | native Tat | p24 |
| Mouse 1 | d72 | 1.2 | 1 | 1.1 | 1 |
| Mouse 2 | d72 | 1 | 1.2 | 1 | 1.1 |
| Mouse 3 | d72 | 1.1 | 1.1 | 1 | 1 |

TABLE 11
Mice immunized with the toxoid Tat:

| | | Gp160 | native Tat | gp160 + native Tat | p24 |
|---|---|---|---|---|---|
| Mouse 4 | d72 | 1.2 | 10 | 9 | 1 |
| Mouse 5 | d72 | 1 | 8 | 10 | 1.2 |
| Mouse 6 | d72 | 1.1 | 9 | 8 | 1.1 |

TABLE 12
Mice immunized with the gp160-toxoid Tat conjugate:

| | | Gp160 | native Tat | gp160 + native Tat | p24 |
|---|---|---|---|---|---|
| Mouse 7 | d72 | 8 | 10 | 9 | 1 |
| Mouse 8 | d72 | 9 | 8 | 10 | 1 |
| Mouse 9 | d72 | 11 | 10 | 9 | 1 |

The splenocytes of the mice immunized with the gp160-toxoid Tat conjugate or the toxoid Tat proliferate, when they are activated, in vitro, with the immunogens used during the immunization.

### Example 7

### Immunogenic Activity of the [Tat Peptides (1-15;46-60)] SIAB-[Toxoid Tat] Conjugate

A. Material and Methods

The immunogenic activity of the Tat-toxoid Tat peptides compared to that of the toxoid Tat alone and their lack of toxicity have been determined in a 18-20 g Balb c mouse according to the protocols described in example 6.

B. Results:

1—Lack of Toxicity of the Superimmunogen in vivo and in vitro

The mice being immunized both by the Tat peptide-toxoide Tat preparation and by the toxoid Tat alone do not show any clinical sign and no anatomic wound. The immunosuppression test shows that the 100 ng/ml doses at 3 μg/ml of peptides-toxoid Tat do not decrease the lymphocyte proliferation.

None of the 3 mice immunized with 100 μg of the preparation shows any toxicity signs (temperature, cutaneous disorders, systemic or regional signs) for the 7 days following the injection.

2-Humoral Response

The humoral response is measured by the presence in the serum of antibodies of the IgG type directed against the native Tat recombinant protein, as measured by ELISA and expressed in titration (opposite of the dilution giving an optical density higher than 0.3)

TABLE 13

Titration

| | |
|---|---|
| d-2 | D72 |

control mouse:

| | | |
|---|---|---|
| mouse 1 | <500⁻¹ | <500⁻ |
| mouse 2 | <500⁻¹ | <500⁻¹ |
| mouse 3 | <500⁻¹ | <500⁻¹ |

mice immunized with the toxoid Tat:

| | | |
|---|---|---|
| mouse 4 | <500⁻¹ | 32,000⁻¹ |
| mouse 5 | <500⁻¹ | 48,000⁻¹ |
| mouse 6 | <500⁻¹ | 48,000⁻¹ |

mice immunized with the peptide Tat-toxoid Tat:

| | | |
|---|---|---|
| mouse 7 | <500⁻¹ | 64,000⁻¹ |
| mouse 8 | <500⁻¹ | 64,000⁻¹ |
| mouse 9 | <500⁻¹ | 64,000⁻¹ |

The mice immunized with the Tat peptide-toxoid Tat conjugate have antibody titration of the IgG anti-Tat type higher than those of the mice immunized with the toxoid Tat alone.

The neutralizing activity of such antibodies has been measured via the Cat assay. The results are given in neutralization %.

TABLE 14
Mice immunized with the toxoide Tat:

Neutralization %

| | | 1/50 | 1/100 | 1/200 | 1/400 |
|---|---|---|---|---|---|
| mouse 4 | d-2 | 0 | 0 | 0 | 0 |
| | d72 | 75 | 50 | 25 | 20 |
| mouse 5 | d-2 | 0 | 0 | 0 | 0 |
| | d72 | 75 | 60 | 30 | 20 |
| mouse 6 | d-2 | 0 | 0 | 0 | 0 |
| | d72 | 75 | 60 | 30 | 20 |

TABLE 15
Mice immunized with the Tat peptide - toxoid Tat conjugate

| | | 1/50 | 1/100 | 1/200 | 1/400 |
|---|---|---|---|---|---|
| mouse 7 | d-2 | 0 | 0 | 0 | 0 |
| | d72 | 100 | 100 | 50 | 25 |
| mouse 8 | d-2 | 0 | 0 | 0 | 0 |
| | d72 | 100 | 100 | 75 | 50 |
| mouse 9 | d-2 | 0 | 0 | 0 | 0 |
| | d72 | 100 | 100 | 60 | 30 |

The antibodies induced by the Tat peptide-toxoid Tat conjugate have a higher neutralizing power than that induced by the toxoid Tat.

### Example 8

### Immunogenic Activity of the E7-SIAB-VEGF conjugate

A. Materials and Methods

The immunogenic activity of the E7-SIAB-VEGF conjugate compared to that of the VEGF and of E7 protein and its lack of toxicity have been determined in a 6 week-old black C57 6 mouse according to the protocols as described in example 6.

B. Results:

1. Lack of Toxicity of the Superimmunogen in vivo and in vitro

The mice immunized as well as by the E7-SIAB-VEGF preparation as by E7 protein or by the VEGF do not show any clinical sign and no anatomic wound. The immunosuppression test shows that the 100 ng/ml doses at 3 μg/ml of the E7-SIAB-VEGF conjugate do not decrease the lymphocyte proliferation.

None of the 3 mice immunized with 100 μg of the preparation does show any toxicity signs (temperature, cutaneous disorders, systemic or regional signs) for the 7 days following the injection.

2. Humoral Response

The humoral response is measured by the presence in the serum of antibodies of the IgG type raised against E7 protein and against the VEGF and, measured via ELISA and expressed in titration (opposite of the dilution giving an optical density higher than 0.3).

TABLE 16
Control mouse:

| | Titration | d-2 | d72 |
|---|---|---|---|
| mouse 1 | E7 | <500⁻¹ | <500⁻¹ |
| | VEGF | <500⁻¹ | <500⁻¹ |
| mouse 2 | E7 | <500⁻¹ | <500⁻¹ |
| | VEGF | <500⁻¹ | <500⁻¹ |
| mouse 3 | E7 | <500⁻¹ | <500⁻¹ |
| | VEGF | <500⁻¹ | <500⁻¹ |

TABLE 17
Mice immunized with the VEGF:

| | | | |
|---|---|---|---|
| mouse 4 | E7 | <500⁻¹ | <500⁻¹ |
| | VEGF | <500⁻¹ | <1000⁻¹ |
| mouse 5 | E7 | <500⁻¹ | <500⁻¹ |
| | VEGF | <500⁻¹ | 1,500⁻¹ |
| mouse 6 | E7 | <500⁻¹ | <500⁻¹ |
| | VEGF | <500⁻¹ | 500⁻¹ |

TABLE 18
mice immunized with E7:

| | | | |
|---|---|---|---|
| mouse 7 | E7 | <500⁻¹ | 48,000⁻¹ |
| | VEGF | <500⁻¹ | <500⁻¹ |
| mouse 8 | E7 | <500⁻¹ | 64,000⁻¹ |
| | VEGF | <500⁻¹ | <500⁻¹ |
| mouse 9 | E7 | <500⁻¹ | 48,000⁻¹ |
| | VEGF | <500⁻¹ | <500⁻¹ |

TABLE 19
Mice immunized with the E7-SIAB-VEGF conjugate:

| | | | |
|---|---|---|---|
| mouse 10 | E7 | <500⁻¹ | 48,000⁻¹ |
| | VEGF | <500⁻¹ | 48,000⁻¹ |
| mouse 11 | E7 | <500⁻¹ | 64,000⁻¹ |
| | VEGF | <500⁻¹ | 32,000⁻¹ |
| mouse 12 | E7 | <500⁻¹ | 48,000⁻¹ |
| | VEGF | <500⁻¹ | 48,000⁻¹ |

The mice immunized with the E7-SIAB-VEGF conjugate have higher antibody titrations of the IgG anti-VEGF type than those of mice immunized with the VEGF alone, whereas their anti-E7 response remains identical to that obtained in mice immunized with E7 alone.

3. Cell Response:

3.1 Proliferation of the Phenocytes of Immunized Mice (CMI)

The splenocytes of the immunized mice and the control mice are isolated and then cultivated in round-bottomed wells of a micro-culture plate at the rate of 100,000 cellules/well in the presence of native E7. The cell culture is continued at 37° C. in a humid atmosphere loaded with 5% of CO2 for 6 days. 18 hours before the end of the incubation, 0.5 μCi of thymidine per well are added. The intensity of the immune response is proportional to the proliferation index Ip.

Ip=cpm (strokes per minute) for the given antigen/control cpm

TABLE 20
Control mouse:

| | |
|---|---|
| mouse 1 d72 | 1.2 |
| mouse 2 d72 | 1 |
| mouse 3 d72 | 1.1 |

Mice immunized with the VEGF:

| | |
|---|---|
| mouse 4 d72 | 1.2 |
| mouse 5 d72 | 1 |
| mouse 6 d72 | 1.1 |

Mice immunized with the E7:

| | |
|---|---|
| mouse 7 d72 | 6 |
| mouse 8 d72 | 10 |
| mouse 9 d72 | 8 |

Mice immunized with the E7-SIAB-VEGF conjugate:

| | |
|---|---|
| mouse 10 d72 | 7 |
| mouse 11 dj72 | 10 |
| mouse 12 d72 | 9 |

The splenocytes of mice immunized with the E7-SIAB-VEGF conjugate, proliferate, when they are activated, in vitro, with E7 protein.

### Example 9

### Immunogenicity of the Betv1a-SIAB-IL4(murine) Conjugate

A. Materials and Methods

The immunogenic activity of the Betv1a-SIAB-II-4 (murine) conjugate compared to that of the Betv1a and of IL4 (murine) and its lack of toxicity have been determined in a 18-20 g Balb c mouse according to the protocols as described in example 6.

B. Results:

1-Lack of Toxicity of the Superimmunogen in vivo and in vitro

The mice immunized as well as by the BetV1a-SIAB-IL4 conjugate as by IL4 alone do not show any clinical sign and no anatomic wound. The immunosuppression test shows that the 100 ng/ml doses at 3 μg/ml of the Betv 1 a-SIAB-IL4 conjugate do not decrease the lymphocyte proliferation.

None of the 3 mice immunized with 100 μg of the preparation shows any toxicity signs (temperature, cutaneous disorders, systemic or regional signs) for the 7 days following the injection.

2. Humoral Response

The humoral response is determined by the presence in the serum of antibodies of the IgG type raised against the Betv1a and IL4, as measured by ELISA and expressed in titration (opposite of the dilution giving an optical density higher than 0.3)

TABLE 21

| Control mouse: | Titration | d-2 | D72 |
|---|---|---|---|
| mouse 1 | Betv1a | <500⁻¹ | <500⁻¹ |
| | IL4 | <500⁻¹ | <500⁻¹ |
| mouse 2 | Betv1a | <500⁻¹ | <500⁻¹ |
| | IL4 | <500⁻¹ | <500⁻¹ |
| mouse 3 | Betv1a | <500⁻¹ | 500⁻¹ |
| | IL4 | <500⁻¹ | <500⁻¹ |

TABLE 22
Mice immunized with Betv1a:

| | Titration | d-2 | D72 |
|---|---|---|---|
| mouse 4 | Betv1a | <500⁻¹ | 48,000⁻¹ |
| | IL4 | <500⁻¹ | <500⁻¹ |
| mouse 5 | Betv1a | <500⁻¹ | 64,000⁻¹ |
| | IL4 | <500⁻¹ | <500⁻¹ |
| mouse 6 | Betv1a | <500⁻¹ | 64,000⁻¹ |
| | IL4 | <500⁻¹ | <500⁻¹ |

Mice immunized with IL4:

| | Titration | d-2 | D72 |
|---|---|---|---|
| mouse 7 | Betv1a | <500⁻¹ | <500⁻¹ |
| | IL4 | <500⁻¹ | 500⁻¹ |
| mouse 8 | Betv1a | <500⁻¹ | <500⁻¹ |
| | IL4 | <500⁻¹ | 1,500⁻¹ |
| mouse 9 | Betv1a | <500⁻¹ | <500⁻¹ |
| | IL4 | <500⁻¹ | 1,000⁻¹ |

TABLE 23
Mice immunized with the Betv1a-SIAB-IL4 conjugate:

| | Titration | d-2 | D72 |
|---|---|---|---|
| mouse 10 | Betv1a | <500⁻¹ | 48,000⁻¹ |
| | IL4 | <500⁻¹ | 48,000⁻¹ |
| mouse 11 | Betv1a | <500⁻¹ | 64,000⁻¹ |
| | IL4 | <500⁻¹ | 32,000⁻¹ |
| mouse 12 | Betv1a | <500⁻¹ | 48,000⁻¹ |
| | IL4 | <500⁻¹ | 48,000⁻¹ |

The mice immunized with the Betv1a-SIAB-II-4 conjugate have higher antibody titrations of the IgG anti-IL4 type that those of mice immuneized with IL4 alone, whereas their anti-Betv1a response remains identical to that obtained in mice immuneized with the Betv1a alone.

### REFERENCES

Adler H L, McCurdy M A, Kattan M W, Timme T L, Scardino P T, Thompson T C. Elevated levels of circulating interleukin-6 and transforming growth factor-beta-1 in patients with metastatic prostatic carcinoma. J Urol 1999;161:182-7

Aucouturier J et al, Adjuvants designed for veterinary and human vaccines. Vaccine 2001 19:2666-72

Baras B. et al, Single-dose mucosal immuneization with biodegradable microparticles containing a Schistosoma mansoni antigen. Infect Immune. (1999) 67:2643-8)

Basak A, Boudreault A, Chen A, Chretien M, Seidah N G, Lazure C., Application of the multiple antigenic peptides (MAP) strategy to the production of prohormone convertases antibodies: synthesis, characterization and use of 8-branched immunogenic peptides.: J Pept Sci 1995 Nov-Dec;1(6):385-95

Chen et al., 1987, Mol. Cell. Biol., 7: 2745-2752.

Cowan et al, Induction of TNF alpha in human neuronal cells by extracellular human T-cell lymphotropic virus Type 1 Tax1, Journal of virology, 1997, 6982-6989.

Ferreira F K et al, 1993, Purification and characterization of recombinant Bet v1, the major Birch pollen allergen: immunological equivalence to natural Bet v1; J. Biol. Chem., 268: 19574.

Flotte et al., 1992, Am. J. Respir. Cell Mol. Biol., 7: 349-356.

Fraley et al., J.Biol.Chem. 255 (1980) 10431

Gopal, 1985, Mol. Cell. Biol., 5: 1188-1190.

Graham et al., 1973, Virology, 52: 456-457.

Harland et al., 1985, J. Cell. Biol., 101: 1094-1095.

Huygen et al., 1996, Nature Medicine, 2(8):893-898

Karayiannakis A J et al, Serum levels of tumor necrosis factor-alpha and nutritional status in pancreatic cancer patients. Anticancer Res 2001,21:1355-8

Le Buanec et al, HPV-16 E7 but not E6 oncogenic protein triggers both cellular immunosuppression and angiogenic processes. Biomed Pharmacother. 1999;53: 424-31.

McLaughlin B A et al., 1996, Am. J. Hum. Genet., 59: 561-569.

Morgenstern J P et al, Amino acid sequence of Feld1, the major allergen of the domestic cat: protein sequence analysis and cDNA cloning, PNAS, 1991,88: 9690

Mori N et al, Interleukine-10 gene expression in adult t-cell leukaemia, Blood, 1996, 103545.

Nicolau C. et al., 1987, Methods Enzymol., 149:157-76.

Potter et al., 1984, Proc Natl Acad Sci U S A.;81(22):7161-5

Samulski et al., 1989, J. Virol., 63: 3822-3828.

Sementchenko V I, Schweinfest C W, Papas T S, Watson D K., ETS2 function is required to maintain the transformed state of human prostate cancer cells. Oncogene 1998;17:2883-8

Skaugrud O et al, Biomedical and pharmaceutical applications of alginate and chitosan. Biotechnol Genet Eng Rev 1999;16:2340

Tacson et al., 1996, Nature Medicine, 2(8):888-892.

Tovey E R et al, Mite faeces are a major source of house dust allergens. Nature, 1981.289: 592-593.

Tur-Kaspa et al, 1986, Mol. Cell. Biol., 6: 716-718.

Yoshiji H et al, Expression of vascular endothelial growth factor, its receptor, and other angiogenic factors in human breast cancer. Cancer Res 1996, 56:2013-6

Zagury D et al., Interferon alpha and Tat involvement in the immunosuppression of uninfected T cells and C-C chemokine decline in AIDS. Proc Natl Acad Sci U S A 1998;95: 3851-6

Zusman I, Sandler B, Gurevich P, Zusman R, Smirnoff P, Tendler Y, Bass D, Shani A, Idelevich E, Pfefferman R, Davidovich B, Huszar M, Glick J. Comparative study of the role of serum levels of p53 antigen and its tumor cell concentration in colon cancer detection. Hum Antibodies Hybridomas. (1996)

1. The use of a composite superimmunogen comprising two distinct polypeptides physically linked one to another, both polypeptides respectively consisting in:
(a) a first immunogenic polypeptide inducing a cell immune reaction, or a cell and humoral immune reaction, towards an inert or a living cell, microbial or particulate pathogenic antigenic structure;
(b) a second immunogenic polypeptide inducing the production of neutralizing or blocking antibodies towards a local circulating protein of the stroma selected amongst a cytokin factor or a cell regulation factor with immunotoxic or angiogenic properties, such a factor being able either to be produced by cancer cells, virus infected cells or stroma cells, including the lymphocytes T and cells having the antigen (APC), or to be induced by pathogenic, including allergenic, particulate structures,
for obtaining a drug with an anti-cancer, anti-viral or anti-allergic action inducing a mucosal or systemic immunity both towards the pathogenic antigenic structure and the local circulating protein of the stroma.

2. A use according to claim 1, characterized in that the first immunogenic polypeptide (a) is selected amongst (i) an immunogenic protein selectively expressed by cancer cells, selectively expressed by virus infected cells or constitutive of an allergenic pathogenic structure, if need be detoxicated, and (ii) a protein derived from the protein (i).

3. A use according to any of claims 1 or 2, characterized in that the immunogenic polypeptide (b) is selected amongst (i) the local circulating protein of the stroma, if need be detoxicated, and (ii) a protein derived from protein (i).

4. A use according to any of claims 1 to 3, characterized in that the first immunogenic polypeptide (a) is selected amongst the proteins of the HIV1 immunogens, immunogenic fragments of such proteins or a protein being derived therefrom.

5. A use according to any of claims 1 to 3, characterized in that the first immunogenic polypeptide (a) is selected amongst the antigens of TAA or TSA tumors, immunogenic fragments of such proteins or an immunogenic protein being derived therefrom.

6. A use according to any of claims 1 to 3, characterized in that the first immunogenic polypeptide (a) is selected amongst allergenic Betv1a, Der p 1 and Fel d 1 proteins, an immunogenic fragment of such proteins or also an immunogenic protein being derived therefrom.

7. A use according to any of claims 1 to 3, characterized in that the polypeptide (a) and the polypeptide (b) are selected amongst:
a) for preventing or treating AIDS:
polypeptide (a): gp160, p24, p17, nef, or Tat proteins of the HIV1 virus, detoxicated or stabilized if required, immunogenic fragments of such proteins or also an immunogenic protein being derived therefrom;
polypeptide (b): Tat, IFNα, and TGFβ proteins, detoxicated if required, immunogenic fragments of such proteins or an immunogenic protein being derived therefrom;
b) for preventing or treating neck of the womb cancer:
polypeptide (a): L1, L2 and E7 proteins of the papillomavirus, preferably of a papillomavirus from strain 16 or 18, detoxicated or stabilized if required, immunogenic fragments of such proteins or also an immunogenic protein being derived therefrom;
polypeptide (b): E7, IFNα, TGFβ, TNFα and VEGF proteins, detoxicated or stabilized if required, immunogenic fragments of such proteins or an immunogenic protein being derived therefrom;
c) for preventing or treating ATL leukemia induced by the HTLV1 or 2 viruses:
polypeptide (a): gp61 and Tax proteins of the HTLV1 or 2 viruses, detoxicated if required, immunogenic fragments of such proteins or also an immunogenic protein being derived therefrom;
polypeptide (b): Tax, IL10, IFNα or TGFβ proteins, detoxicated, immunogenic fragements of such proteins or also a protein being derived therefrom;
d) for preventing or treating colon cancer:
polypeptide (a): CEA and p53 proteins, detoxicated if required, immunogenic fragments of such proteins or also an immunogenic protein being derived therefrom;
polypeptide (b): TGFβ, IL10, p53, FasL and VEGF proteins, detoxicated, immunogenic peptidic fragments of such proteins or also an immunogenic protein being derived therefrom;
e) for preventing or treating breast cancer:
polypeptide (a): Di12 protein, immunogenic fragments of such a protein or also a protein being derived therefrom;
polypeptide (b): TGFβ, TNFα and VEGF proteins, detoxicated if required, immunogenic fragments of such proteins or also an immunogenic protein being derived therefrom;
f) for preventing or treating pancreas cancer:
polypeptide (a): CaSm protein, detoxicated if required, immunogenic fragments of such a protein or also an immunogenic protein being derived therefrom;
polypeptide (b): VEGF and TNFα proteins, detoxicated or stabilized if required, immunogenic fragments of such proteins or also an immunogenic protein being derived therefrom;
g) for preventing or treating prostate cancer:
polypeptide (a): OSA and ETS2 proteins, detoxicated if required, immunogenic fragments of such proteins or also an immunogenic protein being derived therefrom;
polypeptide (b): IL6 and TGFβ proteins, detoxicated if required, immunogenic fragments of such proteins or also an immunogenic protein being derived therefrom;

8. A use according to claim 6, characterized in that the polypeptide (b) is selected amongst the IL4 and IL5 proteins, an immunogenic fragment of such proteins or an immunogenic protein being derived therefrom.

9. A use according to claim 7, characterized in that the composite superimmunogen is selected amongst:
the composite superimmunogen (a) gp160—(b) Tat toxoid;
the composite superimmunogen (b) Tat peptide [1-15;46-60]—(a) gp160;
the composite superimmunogen (a) Tat toxoid—(b) IFNα;
the composite superimmunogen (a) Tat toxoid—(b) Tat peptide [1-1 5;46-60].

10. A use according to claim 7, characterized in that the composite superimmunogen is selected amongst:
the composite superimmunogen (a) L1—(b) E7;
the composite superimmunogen (a) E7—(b) VEGF;

11. A use according to any of claims 1 to 3 and 6, characterized in that the composite superimmunogen is (a) Betv1a—(b) IL4.

12. A use according to any of claims 1 to 11, characterized in that the polypeptides (a) and (b) are directly covalently linked with one another.

13. A use according to any of claims 1 to 11, characterized in that the polypeptides (a) and (b) are separated from one another within the composite superimmunogen by a spacer chain.

14. A use according to claim 13, characterized in that the spacer chain comprises a linear spacer peptide.

15. A use according to claim 13, characterized in that the peptidic chain comprises a branched spacer peptide.

16. A use according to claim 13, characterized in that the spacer chain is a chain of the SIAB or SMMC type.

17. A use according to claim 16, characterized in that the composite superimmunogen is selected amongst:
the composite superimmunogen (a) E7—SIAB—(b) VEGF; and
the composite superimmunogen (a) Betv1a—SIAB—(b) IL4.

18. A use according to any of claims 1 to 8, characterized in that the polypeptide (a) and the polypeptide (b) are both immobilized on one single nanoparticle, or embedded within one single microparticle or within one single nanoparticle.

19. A composite superimmunogen comprising comprising two distinct immunogenic polypeptides, physically linked one to the other, both polypeptides respectively consisting in:
(a) a first immunogenic polypeptide inducing a cell immune reaction, or a cell and humoral immune reaction, towards an inert or a living cell, microbial, or particulate pathogenic antigenic structure;
(b) a second immunogenic polypeptide inducing the production of neutralizing or blocking antibodies towards a local circulating protein of the stroma selected amongst a cytokin factor or a cell regulation factor with immunotoxic or angiogenic properties, such a factor being able either to be produced by cancer cells, virus infected cells or stroma cells, including the lymphocytes T and cells having the antigen (APC), or to be induced by pathogenic, including allergenic, particulate structures.

20. A composite superimmunogen according to claim 19, characterized in that it consists in the (a) gp160—(b) toxoid Tat composite.

21. A composite superimmunogen according to claim 19, characterized in that it consists in the (a) toxoid Tat—(b) IFNα conjugate.

22. A composite superimmunogen according to claim 19, characterized in that it consists in the (a) E7—SIAB—(b) VEGF conjugate.

23. A composite superimmunogen according to claim 19, characterized in that it consists in the (a) BetV1a—SIAB—(b) IL-4 conjugate.

24. A nucleic acid comprising a sequence coding a composite superimmunogen according to any of claims 19 to 23.

25. An expression cassette comprising a polynucleotide coding a composite superimmunogen according to claim 19, under the control of a functional regulating polynucleotide in a mammal, and more specifically in a human.

26. A recombinant vector comprising a nucleic acid according to claim 24 or an expression cassette according to claim 25.

27. A use of a nucleic acid according to claim 24, of an expression cassette according to claim 25 or a recombinant vector according to claim 26 for obtaining a drug having an anti-cancer, anti-viral or anti-allergic action.

28. An immunogenic composition characterized in that it comprises an immunologically efficient amount of a composite superimmunogen according to any of claims 19 to 23, in association with one or more physiologically compatible excipients.

29. A vaccine, characterized in that it comprises, as an active, a composite superimmunogen according to any of claims 19 to 23, in association with one or more physiologically compatible excipients.

30. An immunogenic composition characterized in that it comprises a therapeutically efficient amount of a nucleic acid according to claim 24, an expression cassette according to claim 25 or a recombinant vector according to claim 26.

31. A vaccine characterized in that it comprises a therapeutically efficient amount of a nucleic acid according to claim 24, an expression cassette according to claim 25 or a recombinant vector according to claim 26.
